SEQUENCE LISTING (13 sequences)

SEQ ID NO: 1 - DNA, 135 bp, Mus sp.
atgtctgacaaacccgatatggctgagatcgagaaattcgataagtcgaagttgaagaaaacagaaacgcaagagaaaaatcctctgccttcaaaagaaacaattgaacaagagaagcaagctggcgaatcgtaa

SEQ ID NO: 2 - DNA, 135 bp, Homo sapiens
atgtctgacaaacccgatatggctgagatcgagaaattcgataagtcgaaactgaagaagacagagacgcaagagaaaaatccactgccttccaaagaaacgattgaacaggagaagcaagcaggcgaatcgtaa

SEQ ID NO: 3 - DNA, 135 bp, Artificial
  TB4 DNA antisense
ttacgattcgccagcttgcttctcttgttcaattgtttcttttgaaggcagaggatttttctcttgcgtttctgttttcttcaacttcgacttatcgaatttctcgatctcagccatatcgggtttgtcagacat

SEQ ID NO: 4 - DNA, 33 bp, Artificial
  PCR Primer
cgcggatccagatgtctgacaaacccgatatgg

SEQ ID NO: 5 - DNA, 31 bp, Artificial
  PCR Primer
ccgctcgagttacgattcgccagcttgcttc

SEQ ID NO: 6 - DNA, 32 bp, Artificial
  PCR Primer
cccaagcttaccatggactacccttatgatgt

SEQ ID NO: 7 - DNA, 31 bp, artificial; identical to SEQ ID NO: 5
  PCR Primer

SEQ ID NO: 8 - DNA, 22 bp, artificial
  PCR Primer
aagcagaagaacggcatcaagg

SEQ ID NO: 9 - DNA, 20 bp, Artificial
  PCR Primer
tctttgctcagggcggactg

SEQ ID NO: 10 - DNA, 20 bp, artificial
  PCR Primer
gtaacccgttgaaccccatt

SEQ ID NO: 11 - DNA, 20 bp, artificial
  PCR Primer
ccatccaatcggtagtagcg

SEQ ID NO: 12 - DNA, 20 bp, artificial
  PCR Primer
agttcaggcccactctcctt

SEQ ID NO: 13 - DNA, 20 bp, artificial
  PCR Primer
agtcaccacttgctgctgtg

(19) United States
(12) Patent Application Publication
(10) Pub. No.: US 2011/0123499 A1
(43) Pub. Date: May 26, 2011

(54) CELLULAR DIFFERENTIATION PROMOTION

(75) Inventors: Daniela Merlo, Rome (IT); Cristiana Mollinari, Rome (IT); Ruggero De Maria, Rome (IT); Enrico Garaci, Rome (IT)

(73) Assignee: ISTITUTO SUPERIORE DI SANITA, Rome (IT)

(21) Appl. No.: 12/921,645

(22) PCT Filed: Mar. 11, 2009
(86) PCT No.: PCT/EP2009/002055

§ 371 Date: Nov. 8, 2010

(30) Foreign Application Priority Data
Mar. 11, 2008 (GB) 0804501.5

(51) Int. Cl. A61K 35/12; C12N 5/02; C12N 5/071; A61P 25/28

(52) U.S. Cl. 424/93.7; 435/325; 435/366

(57) ABSTRACT

Neural precursor cells can be encouraged to form mature neural cells twice as quickly in the absence, or reduced expression of, thymosin β4.

The present invention relates to the regeneration of nervous tissue, to progenitor cells of such tissue, and to methods for such regeneration.

Dynamic remodelling of the actin cytoskeleton occurs during cellular migration, differentiation, and cytokinesis {Pollard, 2001 #1} {Pollard, 2003 #2}, and is controlled by an array of actin-binding proteins that act on the dynamic process of filament polymerisation, cross-linking and interaction with cellular membranes {McGough, 1998 #3}

Thymosin β4 (Tβ4), the most abundant member of the thymosin-βs (Tβs) protein family, is regarded as the main G-actin sequestering peptide in the cytoplasm, modulating the availability of actin monomers in a large variety of cells {Safer, 1994 #10} {Huff, 2001 #9} {Sun, 2007 #8} {Erickson-Viitanen, 1983 #7}. Tβ4 shows many pleiotropic effects, being involved in carcinogenesis {Cha, 2003 #6} {Yamamoto, 1994 #4} {Hall, 1991 #5} {Kobayashi, 2002 #69} {Goldstein, 2003 #9}, apoptosis {Choi, 2006 #12} {Sosne, 2004 #13} {Muller, 2003 #14} {Bock-Marquette, 2004 #15}, angiogenesis {Grant, 1999 #16} {Philip, 2004 #17} {Malinda, 1997 #18} wound healing {Malinda, 1999 #19} and regulation of inflammation {Sosne, 2007 #21} {Sosne, 2002 #20}. In the brain, Tβs are highly expressed {Devineni, 1999 #22} and are believed to play a role in the regulation of normal patterns of neurite outgrowth {Dathe, 2004 #23} {Gomez-Marquez, 2002 #24} {Lin, 1990 #25} {Roth, 1999 #36} {Boquet, 2000 #40} {Carpinterio, 1995 #30} {Choe, 2005 #50} {Roth, 1999 #37} {Roth, 1999 #26} {van Kesteren, 2006 #31}. The complex pleiotropic effects of Tβ4 in cells may be linked to its direct action on the actin cytoskeleton, as well as the modulation of signalling pathways controlling the cytoskeleton.

Neurite elongation, branching and pathfinding during brain development are processes highly dependent on the reorganisation and dynamics of the actin cytoskeleton {Bradke, 1999 #28} {Bradke, 2000 #29} {Chen, 2000 #30} {Luo, 2000 #32} {Gallo, 2004 #33}. Cell adhesion molecules represent important cell surface-associated neurite-promoting factors. Among them, N-cadherin, complexed to catenins {Yap, 1997 #34}, provide traction forces and trigger intracellular signalling cascades required for neurite extension {Kiryushko, 2004 #35}.

{Choe, 2005 #47} reported that Tβ4 is enriched in developing neurite processes where actin is required. In cultured Aplysia sensory neurons, mRNA encoding Tβ is one of the most abundant transcripts in neurites {Moccia, 2003 #73}, but it has also been reported that down-regulation of Tβ leads to a significant increase in neurite outgrowth in Lymnaea pedal neurons {van Kesteren, 2006 #31}. In this respect, controversial results have been reported on the neurite promoting activity of Tβs. In fact, in zebrafish, in vitro over-expression of Tβ in regenerating retinal ganglion cells, results in alterations of neurite shape and excessive branching, {Roth, 1999 #27}, and in vivo knockdown of Tβ results in malformation of retinal axon tracts {Roth, 1999 #26}. In cultured cortical and hippocampal neurons, over-expression of Tβ, in particular Tβ15, enhances neurite branch formation through its G-actin sequestering activity {Choe, 2005 #47}.

It is not clear which type of actin cytoskeletal reorganisation (depolymerisation or polymerisation) regulates neurite formation or retraction. It may be that both actin cytoskeletal rearrangements are necessary in different moments and places within the growth cone {Bradke, 1999 #28}. This would justify the opposing results obtained by the over-expression or down-regulation of different actin-binding proteins including Tβ4. It is also disputed as to the effect that modulation of Tβ4 concentration has on the actin cytoskeleton. Tβ4 is generally believed to sequester monomeric G-actin, thus facilitating actin filament depolymerisation {Sanger, 1995 #65}. However, exceptions to this have been reported depending on cell type, levels of Tβ4 expression and other actin-binding partners within the cell {Golla, 1997 #75}.

We have now found that down-regulation of Tβ4 expression affects the neuronal differentiation of mouse embryonic neural progenitor cells (NPCs) in vitro. This mouse model is widely used to identify molecules controlling important processes related to neuronal development.

Thus, in a first aspect, there is provided a neural progenitor cell for implanting in a patient, wherein the cell has been treated to reduce Thymosin β4 (Tβ4) expression.

NPCs for use in accordance with the present invention are preferably from the same species as the patient. It will be appreciated that, while reference is commonly made herein to NPCs in the plural, this includes reference to an NPC in the singular, where appropriate. The patient is preferably human, and the NPCs are preferably obtained from a blood relative of the patient or a close serological match therefor and, more preferably, from the patient him- or her-self. The NPCs can be derived from a foetus or umbilical cord, or any suitable location in the body, but is preferably obtained from the brain, and preferably the brain of the patient. NPCs have been successfully obtained from the hippocampus, subventricular zone and olfactory bulb, for example. It is generally preferred to isolate a multipotent NPC rather than a pluripotent or even omnipotent precursor therefor.

How the NPCs are obtained is not important to the present invention. It is preferred to culture the NPCs in a manner common to the cultivation of other multipotent stem cells in order to obtain a neurosphere. NPCs may be cultured in a medium containing epidermal growth factor (EGF) and basic fibroblast growth factor (bFGF). The neurosphere may then be treated to reduce Tβ4 expression. Such treatment may involve any suitable method to suppress expression of Tβ4. We exemplify the use of antisense DNA herein, and this may be introduced by the use of a suitable expression plasmid or lentivirus for example. It is preferred not to use a retrovirus, although it is possible, as only low levels of transduction are generally observed with retroviruses. Expression vectors and lentiviridae will generally become attenuated and eventually disappear from the system.

Murine thymosin beta 4 cDNA has been deposited and is available, GenBank accession number: NM 021278 (on chromosome X), see SEQ ID No. 1.

5′-ATGTCTGACAAACCCGATATGGCTGAGATCGAGAAATTCGATAAGT
CGAAGTTGAAGAAAACAGAAACGCAAGAGAAAAATCCTCTGCCTTCAAA
AGAAACAATTGAACAAGAGAAGCAAGCTGGCGAATCGTAA-3′

It will be appreciated that human Tβ4 has a very similar structure and can be readily elucidated using antibodies to the murine protein.

Human Thymosin beta 4 (X-linked), GenBank accession number: NM 021109 has the sequence of SEQ ID No. 2:

5′-ATGTCTGACAAACCCGATATGGCTGAGATCGAGAAATTCGATAAGT
CGAAACTGAAGAAGACAGAGACGCAAGAGAAAAATCCACTGCCTTCCAA
AGAAACGATTGAACAGGAGAAGCAAGCAGGCGAATCGTAA-3′

In general, the sequence of Tβ4 is not important, save that interfering nucleotide sequences will generally have a whole or partial antisense sequence to either of the above sequences. An antisense sequence to the murine sequence will generally also be effective in humans, despite mismatches, as it is not necessary for an antisense sequence to be completely complementary to the coding sequence to have a suppressive effect. Indeed, there are only 9 mismatches on 136 bases between the murine and human coding sequences, and the protein sequence is identical.

The DNA antisense sequence used in the accompanying Examples was based on the above sequence, and was that shown in SEQ ID No. 3, although any suitable antisense sequence may be used, as described hereinbelow.

5′-TTACGATTCGCCAGCTTGCTTCTCTTGTTCAATTGTTTCTTTTGAA
GGCAGAGGATTTTTCTCTTGCGTTTCTGTTTTCTTCAACTTCGACTTAT
CGAATTTCTCGATCTCAGCCATATCGGGTTTGTCAGACAT-3′

SEQ ID NO. 3 or a fragment or variant thereof capable of hybridising to TB4, and substantially reducing the expression thereof, is preferred. Hybridisation may be under stringent conditions, preferably 0.165-0.330 [NaCl] (Molar) or 20 to 29 degrees C. below Tm and more preferably 0.0165-0.0330 [NaCl] (Molar) or 5 to 10 degrees C. below Tm. Washing at 6×SSC is also preferred. The fragment or variant may also comprise a sequence having at least 70%, more preferably at least 80%, more preferably at least 85%, more preferably at least 90%, more preferably at least 95%, and more preferably at least 99% sequence homology to SEQ ID NO. 3. This may be assessed using the BLAST program, for instance. The antisense may be any polynucleotide, including DNA, RNA or a mixture thereof. The above also applies to any other sequence mentioned herein.

Suitable treatment may also comprise the use of miRNA and/or siRNA to silence or reduce expression of Tβ4 by interfering with the Tβ4 mRNA, and these RNAs may be encoded by a suitable expression vector.

Antibodies, and fragments thereof, may also be used, but it is generally preferred to use nucleic acid sequences. Such sequences need only interfere with expression, and will generally be antisense to the Tβ4 coding sequence, whether on the genome or RNA. As it is only necessary to recognise the sense sequence, it is not essential that the antisense sequence match the sense sequence base for base. The antisense sequence may vary from full length antisense, to shorter sequences of 10 to about 40, more preferably about 15 to about 30 bases, and particularly preferably herein, about 22-23 nucleotide siRNA, which serves to guide cleavage of target Tβ4 mRNA. The RNA may comprise one sense sequence and one antisense sequence (complementary) separated by a nonsense sequence so that a loop is created. This miRNA, which after base pairing between the mature miRNA and its target Tβ4 mRNA, thereby leads to Tβ4 mRNA cleavage or to Tβ4 mRNA translation inhibition,

Expression vectors and plasmids may contain promoters that are selectively active in neural cells so as not to reduce expression in other cells, should the vector transfect another cell type, but this is generally unlikely in vivo.

Tβ4 expression is reduced, and this may be by between about 50% and 100% of the amount of expression usually observed in NPCs not so treated. It is generally preferred to reduce expression to very low levels, such as less than 10%, and preferably less than 5%, and levels of substantially 0%, where Tβ4 is not detectable, as illustrated hereinbelow, are preferred.

Reduction of Tβ4 expression is associated with an increase in N-cadherin expression (a 1.6-fold increase has been observed) and an increase in β-catenin expression (a 1.8-fold increase has been observed). Thus, increased expression of either, or both, of N-cadherin and β-catenin may be taken as evidence of transfection in neurospheres.

The NPCs are implantable in a patient, and will generally be stored or cultured separately from the patient until needed. Such storage and culture may be by any means known in the art for the storage and culture of this type of multipotent stem cell.

Implantation may typically be by injection or surgical technique in an area that requires repair of damaged or compromised nervous tissue. It is preferred that the NPCs of the invention be used to treat conditions of the brain, such as brain damage or neurodegenerative disorders, such Alzheimer's, Parkinson's, stroke and other conditions where neurons have been damaged, destroyed or killed.

Administration of the NPCs to the desired area may be in combination with a suitable nutrient, carrier or structural framework to encourage growth and differentiation, since suitable conditions will not often be present in situ. The NPCs of the invention may also be grown and started down the differentiation pathway prior to implantation, but it is preferred only to start the process for a short while prior to implantation, as it is preferred that the growing neuron adapt to its environment.

It will also be appreciated that neurospheres may be implanted directly in the patient and treated in situ to suppress Tβ4 expression, or the neurospheres may be prepared with the treatment in a syringe prior to injection. It will also be appreciated that pre-existing, or endogenous, NPCs may be treated in situ to suppress Tβ4 expression, and that such treatment forms a part of the present invention. The present invention contemplates such methods, but it is generally preferred to incubate the NPCs, preferably in the form of neurospheres, with the treatment to reduce expression of Tβ4 in order to stabilise incorporation of the treatment. This may be done in the presence of EGF and/or bFGF, preferably both, in order to prevent differentiation, with removal of these growth factors being achieved by the simple expedient of implantation in the patient with the subsequent resulting dilution and removal by the patient's circulation.

Treatment may be verified by the presence of a suitable marker, such as a fluorescent protein. GFP may be used, and the EGFP reporter gene, optionally under the control of the PGK promoter, may be used, for example. Transfected NPCs may then be selected in accordance with whether they fluoresce under the selected conditions.

It will be appreciated that the present invention extends to a method for the treatment of a patient requiring neuroregeneration comprising administering an NPC as defined herein to the area of the patient requiring neuroregeneration. Preferred conditions for treatment are those identified above. Also provided is the use of the present NPCs in the manufacture of a medicament for the stimulation of neuroregeneration. It will be appreciated that where reference is made to treatment neuroregeneration, this may also apply to treatment of neurodegeneration, fibre regeneration and tissue repair or the treatment of neruronal, for instance spinal, damage.

We have now found that Tβ4 is functionally involved in the neuronal differentiation of embryonic NPCs, controlling neurite extension and the availability of ion-channels probably influencing the N-cadherin/ERK signalling pathway.

Thymosin β4 (Tβ4) is a 43-amino acid actin-binding peptide highly expressed in the developing brain of different organisms, and its expression tightly correlates with migration and neurite extension in developing neurons. We have found that the down-regulation of Tβ4 expression affects growth and differentiation of murine embryonic neural progenitor cells (NPCs), and can be effected using an antisense strategy mediated by lentiviral vectors, for example. In undifferentiated cultural conditions, Tβ4 antisense-transduced neurospheres show increased expression of N-cadherin/β-catenin, while maintaining an unaltered proliferative capacity, sphere morphology and expression of the stem cell marker nestin.

When a differentiating culture medium is applied, the number of neurons derived from Tβ4 antisense-transduced NPCs doubles. Moreover, Tβ4 antisense neurons have significantly enhanced neurite outgrowth and a higher number of major neurites, accompanied by increased N-cadherin/β-catenin expression and extracellular-signal-regulated kinase (ERK) activation. Electrophysiological analysis shows that neurons with down-regulated expression of Tβ4 respond in advance and with an increased amplitude of kainate-induced currents associated with α-amino-3-hydroxy-5-methylsoxazole-4-proprionate (AMPA) receptor GluR2/3 subunit increment, thus indicating a more rapid maturation of these neurons.

In order to demonstrate the effects of reducing Tβ4 expression, NPCs were stably transduced with lentiviral vectors in order to over-express either the Tβ4 antisense or just the empty vector as control, and cultured as neurospheres or under differentiating conditions. In undifferentiating conditions, Tβ4 antisense-transduced neurospheres show a stronger expression of N-cadherin/β-catenin but similar morphology and proliferation capacity, as compared with control neurospheres. Under differentiating conditions, neurons with a down-regulated Tβ4 expression were higher in number, had a significant increase in neurite outgrowth and an elevated number of major neurites, starting from the initial days of differentiation. Induction of neurite growth is accompanied by an increase in expression of both N-cadherin and β-catenin, as well as extracellular-signal-regulated kinase (ERK) activation. In addition, neurons derived from Tβ4 antisense-transduced NPCs demonstrated accelerated differentiation and a glutamate-α-amino-3-hydroxy-5-methylsoxazole-4-proprionate (AMPA) current of increased amplitude in response to kainate administration, associated with an increased expression of AMPA receptors GluR2/3 subunits.

Down-regulation of Tβ4 levels in NPCs cultured as neurospheres and under differentiating conditions has shown that: (i) neurospheres transduced with the Tβ4-antisense do not show significant differences in terms of sphere morphology, stem cell marker expression, and cell cycle profile, although biochemical analysis revealed an up-regulation of the adhesion molecule N-cadherin and its cytoplasmic partner β-catenin; (ii) Tβ4-antisense-transduced NPCs differentiate to provide double the number of neurons showing a higher number of prominent neurites and significantly enhanced neurite outgrowth; (iii) differentiating NPCs transduced with Tβ4-antisense have an increased expression of N-cadherin/β catenin and ERK activation; (iv) neurons derived from Tβ4-antisense-transduced NPCs show increased surface exposure of AMPA receptors.

Controversial results have been reported on the role of Tβ4 in cellular proliferation {Cha, 2003 #6} {Wang, 2003 #68} {Kobayashi, 2002 #69} {Huang, 2006 #41}. Indeed, several studies indicate that Tβ4 is regulated by cell proliferation but is not a cell cycle-regulated gene {Zalvide, 1995 #64} {Otero, 1993 #103}. Although mitosis is highly dependent on actin dynamics, the involvement of Tβ4 in the process of citokinesis has not been well elucidated {Sanger, 1995 #65} {Otero, 1993 #103}. Tβ4 antisense-transduced neurospheres divide normally and do not show accumulation of cells with two or more nuclei, which usually indicates a cytokinetic block. This result was unexpected. Since actin dynamic has a fundamental role in cellular division, Tβ4 down-regulation would have been expected to affect cellular division and, more specifically, the phase of cytokinesis during which a ring of actin has to be formed. Without being bound by theory, it is possible that NPCs, growing in suspension, employ alternative mechanisms of mitosis completion {Uyeda, 2004 #66} thus being less sensitive to variation of expression of Tβ4.

In neurons derived from NPCs, we found that Tβ4 is mostly localised in neuronal processes and growth cones. This subcellular localisation is consistent with Tβ4 playing a role in regulating neurite outgrowth in NPCs. In agreement with the above-mentioned studies, we found that differentiating NPCs develop a higher number of prominent neurites with an increased length, when Tβ4 is down-regulated.

Without being bound by theory, it appears likely that, in embryonic NPCs, the effects of Tβ4 antisense are probably mediated by a polymerisation effect, resulting in an enhanced neurite outgrowth, as reported for Lymnaea pedal neurons. Similarly, over-expression of profiling I, which usually promotes actin polymerisation, induces enhanced neurite outgrowth {Lambrechts, 2006 #76}.

Rapid changes in the rearrangements of filamentous actin within particular region of the cell may be important in controlling other additional mechanisms necessary during neuritogenesis, including anchoring and redistribution of cell adhesion molecules within the membrane {Letourneau, 1989 #77}. Thus, it is possible that rapid changes in filamentous actin, induced by alteration of Tβ4 levels, result in a different anchoring of membrane proteins which are needed during neurite growth {Yu, 2003 #78} {Theriot, 1994 #79}. Adhesion molecules such as N-cadherin are involved in forming calcium-dependent cell-cell adhesion, neurite outgrowth and synaptic junctions in the nervous system {Takeichi, 1995 #81} {Bixby, 1990 #82} {Saffell, 1997 #58} {Utton, 2001 #51} {Iwai, 1997 #80} {Tanaka, 2000 #84}. Indeed, we found that the down-regulation of Tβ4 in NPCs induces an increase in the expression of N-cadherin and β-catenin. Since increases in this adhesion complex have been shown to generate neurons with higher neurite output {Otero, 2004 #85}, the observed different morphologies of neurons derived from Tβ4-antisense NPCs may be due to the up-regulation of the adhesion complex. Indeed, another example in which an increased expression of the N-cadherin-mediated cell-cell adhesion is translated into a higher neurite outgrowth is reported by {Chen, 2005 #54}.

It is known that N-cadherin can activate ERKs and induce neurite outgrowth {Perron, 1999 #57}. In addition, pharmacological inhibition of ERK activation strongly inhibits the ability of this adhesion protein to promote neurite growth {Pang, 1995 #87}. Moreover, ERKs play a significant role in neuronal differentiation, initiation of neurite outgrowth and rearrangement of neurites {Sweatt, 2001 #86}. Without being bound by theory, it may be that the phenotype found in Tβ4 antisense neurons is due to the increment of N-cadherin which in turn has supported the activation of ERKs that we observed. However, we cannot exclude that Tβ4-induced actin cytoskeleton remodelling has first activated ERKs and that the sustained activation of ERKs has influenced neurite outgrowth, neuronal fate decision and has increased the expression of the N-cadherin/β catenin adhesion complex.

It may also be that Tβ4 directly influences the various aspects of neuronal differentiation of NPCs by antagonising commitment of progenitors to the neuronal lineage. Indeed, Tβ4 has been detected in the nucleus of cells where it may alter the expression of different genes directly involved in the determination of neuronal fate {Huff, 2004 #88} {Moon, 2006 #89}. Cytochalasin, by depolymerising actin cytoskeleton, is able to change the gene transcription program in Schwann cells in culture {Fernandez-Valle, 1997 #90}. We have also shown that a reduction in Tβ4 facilitates neuronal differentiation of NPCs without increasing proliferation of neural progenitors, but probably by enhancing exit from the cell cycle and having an instructive differentiating effect.

Tβ4 increases AMPA receptors surface exposure in neurons derived from Tβ4-antisense-transduced progenitors. It has been recently demonstrated that N-cadherin is associated with AMPA receptors and increases their surface expression level in neurons {Nuriya, 2006 #91}. This result confirms that actin binding proteins are able to affect ion channel distribution and regulate receptor trafficking in neurons. For example, down-regulation of the Tβ orthologue (Csp24) induces changes in the distribution of IC channels affecting the amplitude of the A-type transient K+ current (I(A)) in Hermissenda sensory neurons {Redell, 2007 #92} {Yamoah, 2005 #93}. The interaction between channels and an actin-binding protein is also supported by studies showing that Kv4.2 current density is substantially larger in filament positive cells as compared with filament negative cells {Petrecca, 2000 #94}. Moreover, the role of the actin network in regulating ion channel localisation and activity has been shown to be an important factor in establishing the electrical properties of neurons {Petrecca, 2000 #94} {Hattan, 2002 #95} {Misonou, 2004 #96}.

Replacement of degenerated neurons by grafting neural progenitors is an important therapeutic strategy to restore lost circuits in many degenerative diseases {Fisher, 1995 #97} {Dunnett, 1995 #99} {Olson, 1997 #100}. Neurite outgrowth from grafted progenitor cells is critical for amelioration of symptoms in many neurodegenerative diseases, and the use of Tβ4 as a factor able to influence neuronal fate, neurite extension and ion channel distribution is important in such therapies.

When the Tβ4 antisense-transduced NPCs were transplanted in vivo into a mouse model of spinal cord injury, they survived and retained their differentiation capability, promoting the recovery of locomotion in injured mice. Locomotory recovery correlated with increased expression of the regeneration-promoting cell adhesion molecule L1 by the grafted Tβ4 antisense-transduced NPCs. This resulted in an increased number of regenerating β-tubulin III-positive axons and in sprouting of serotonergic fibres surrounding and contacting the Tβ4 antisense-transduced NPCs grafted into the lesion site.

The adhesion molecule L1 favours axonal growth in an inhibitory environment (Lemmon et al., 1989; Fransen et al., 1998; Castellani et al., 2002; Dong et al., 2002; Roonprapunt et al., 2003; Xu et al., 2004; Chen et al., 2005a; Zhang et al., 2005), promotes neurite outgrowth and displays survival-promoting effects on cultured central nervous system neurons (Lindner et al., 1983; Lemmon et al., 1989; Chen et al., 1999; Dong et al., 2002; Dong et al., 2003; Rathjen and Rutishauser, 1984). Embryonic stem cells over-expressing L1 support the regrowth of corticospinal tract axons and survive better than non-transfected stem cells in the injured spinal cord of adult mice (Chen et al., 2005a). Similarly, functional recovery and positive effects on damaged 5-HT and corticospinal axons of adult injured mice were reported after injection of an adenovirus expressing human L1 protein (Chen et al., 2007). We also observed an increased number of β-tubulin III-positive fibres travelling close to the grafted Tβ4 antisense-transduced NPCs in the lesioned area. The observation that β-tubulin III-positive fibres were found positive for GAP43, a universal indicator of axonal growth status, underlines that enhanced axonal growth and regeneration occurred after Tβ4 antisense-transduced NPC graft. The robust serotonergic sprouting also demonstrates the potential of grafted Tβ4 antisense-transduced NPCs in promoting regeneration of spared host fibres. Thus, it is possible that Tβ4 antisense-transduced NPCs facilitate axonal regeneration by providing a growth-permitting guiding substrate through stimulation of the production of L1. Interestingly, a direct link between L1 and Tβ4 has recently been shown where Tβ4 enhances L1 expression in a dose-dependent manner, and L1 mediates Tβ4-induced neurite outgrowth and survival in neurons in vitro (Yang et al., 2008).

In short, we have also shown that:

1). Animals grafted with Tbeta4 as-NPCs (NPCs expressing Tβ4 antisense nucleotides) show improved locomotory function in a mouse spinal cord injury model;
2). Transplanted Tbeta4 as-NPCs survive and differentiate in injured mouse spinal cord;
3). Transplanted Tbeta4 as-NPCs overexpress L1;
4). Transplanted Tbeta4 as-NPCs promote an increase in the number of Beta Tubulin III-positive fibres which immunostained with GAP43, a universal indicator of axonal growth status; and
5). Transplanted Tbeta4 as-NPCs promote sprouting of host serotonergic fibres.

The ability of Tβ4 antisense-transduced NPCs to foster a more permissive/hospitable environment for fibres regeneration and tissue repair may have important implications for therapeutic intervention to improve outcome after spinal cord injury.

The invention will now be described in reference to the following non-limiting Examples. Any references herein are herby incorporated by reference to the extent that they do not conflict with the present invention.

### EXAMPLES

### Dissection and Culturing

Telencephalic regions from embryonic day E14 wild type CD1 were dissected and incubated in 0.1% trypsin and 0.05% Dnase in DMEM for 15 min at 37° C. followed by mechanical dissociation. Cells were cultured in the presence of 20 ng/ml of human EGF and 10 ng/ml of human bFGF in DMEM-F12 medium (Euroclone; Irvine, Scotland), containing 2 mM L-glutamine, 0.6% glucose, 9.6 g/ml putrescine, 6.3 ng/ml progesterone, 5.2 ng/ml sodium selenite, 0.025 mg/ml insulin, and 0.1 mg/ml tissue-purified transferrin (Sigma-Aldrich). Floating neurospheres were mechanically dissociated to obtain a single cell suspension every 5-7 days, and they were used from passage 5 to 12 throughout the study. Neurosphere differentiation was induced by withdrawal of EGF and bFGF in the presence of 1% FCS (Gibco) on a matrigel substratum (Beckton and Dickinson). In differentiation conditions neurons were counted every day and the result compared with the total cell number.

### Immunofluorescence and Confocal Microscopy

Cells were fixed for 20 min in 4% paraformaldehyde in PBS and then washed and permeabilised with PBS/0.2% Triton-X. Slides were then incubated in PBS/0.05% Tween 20 containing 3% BSA and the appropriate antibody mixture for 1 h at 37° C. The primary antibodies were rabbit anti-GFAP (1:1000; Chemicon), mouse anti-β-tubulin III (1:500; Chemicon), and mouse anti-MAP2 (1:500; Sigma). After washing, slides were incubated for 30 min at 37° C. with Cy2/Cy3/Cy5-conjugated secondary antibodies (Jackson Immunoresearch). Finally, coverslips were counterstained with Hoechst 33342 (Sigma), mounted with a anti-fading glycerol medium and observed with a confocal microscope (Nikon Instruments Spa, Eclipse TE 200 equipped with a 405 nm diode; and FluoView 300 Olympus). In every experiment, at least 500 cells were counted in 10 different fields to calculate the percentage of neurons. For the length of neurites, 20 neurites/day from 3 independent experiments were measured from the cell body to the tip of the longest process, using TIFF files with ImageJ.

### Lentiviral Production and Transduction

Tβ4 cDNA was subcloned into a modified pcDNA3 (KpnI-XhoI sites) containing an HA-tag in frame with the coding sequence of Tβ4. From this plasmid, the HA-Tβ4 cDNA was subcloned in the antisense orientation, under the control of the CMV promoter of a lentiviral vector which carried the EGFP reporter gene under the control of the PGK promoter. Recombinant lentiviruses were derived by the combined transfection of different plasmids as described by {Ricci-Vitiani, 2004 #104}. The infections were monitored by flow cytometry and cells were sorted for their fluorescence (FACS Vantage, Becton and Dickinson) until we obtained a virtually pure population of transduced cells expressing EGFP alone (empty vector) or the antisense Tβ4.

### Real-Time PCR

Total RNA was transcribed into cDNA using the Superscript II system (Superscript, Invitrogen) and pd(N)6 random nucleotide. Relative quantitative Real-Time PCR was performed in a Real-Time Thermocycler (MX 3000, Stratagene, Milano, Italy) using the Brilliant SYBR Green QPCR Master Mix according to the manufacturer's instructions. All PCR reactions were coupled to melting-curve analysis to confirm amplification specificity. Non-template controls were included for each primer pair to check for any significant levels of contaminants. Specific primers for mouse Tβ4 and 18S rRNA were designed in order to amplify short DNA fragments (110-200 bp in length). Gene-specific primers in the mouse Tβ4 coding sequence were:

| | |
|---|---|
| upstream: | |
| CGCGGATCCAGATGTCTGACAAACCCGATATGG; | SEQ ID No. 4 |
| and | |
| downstream: | |
| CCGCTCGAGTTACGATTCGCCAGCTTGCTTC. | SEQ ID No. 5 |

Primers to detect the exogenous Tβ4 antisense were: upstream in the HA tag (SEQ ID No. 6) CCCAAGCTTACCATGGACTACCCTTATGATGT; and downstream (SEQ ID No. 7) CCGCTCGAGTTACGATTCGCCAGCTTGCTTC.

Primers to detect the expression of the EGFP were:

| | | |
|---|---|---|
| upstream: | AAGCAGAAGAACGGCATCAAGG; | SEQ ID No. 8 |
| and | | |
| downstream: | TCTTTGCTCAGGGCGGACTG. | SEQ ID No. 9 |

18S upstream: SEQ ID No. 10 GTAACCCGTTGAACCCCATT; and downstream: SEQ ID No. 11 CCATCCAATCGGTAGTAGCG.

Tβ4 levels were normalised to the expression of 18S rRNA. The relative quantitation was calculated with the analysis software that accompanied the thermal cycler.

### Single Cell Real-Time PCR

After recording, the cellular cytoplasm was aspirated into a pipette. The content of the pipette was then released into a chilled 0.2 μL thin-walled PCR tube, containing 12 μl of the RT-PCR mixture 1× reverse transcriptase buffer (Life Technologies), 0.5% Nonidet P-40, 5 μm random hexamer primers, 5 U/ml RNasiOUT (Life Technologies), and 0.5 mM each of deoxy (d)-nucleotides. Samples were retrotranscribed using the Superscript II system (Invitrogen). For amplification, half the mixture was used to amplify the MAP2 transcript and the other half 18S rRNA using the Brilliant SYBR Green QPCR Master Mix. Gene-specific primers in the mouse MAP2 coding sequence were:

| | | |
|---|---|---|
| upstream: | AGTTCAGGCCCACTCTCCTT; | SEQ ID No. 12 |
| and | | |
| downstream: | AGTCACCACTTGCTGCTGTG. | SEQ ID No. 13 |

### Western Blotting

Neurosphere or cellular pellets were lysed in RIPA buffer: 150 mM NaCl, 10 mM Tris-HCl, 1 mM EDTA and 1% Triton-X100 and protease inhibitors (Sigma), 1 mM PMSF pH7.4. Samples were resolved in SDS-PAGE gels (13% for Tβ4 detection) and proteins were loaded after measurement with Bradford assay (Biorad). Purified Tβ4 peptide (10 μM) was run as a reference for protein migration. For Tβ4 detection, the acrylamide gel was washed several times in PBS for 1 h and then incubated in 10% glutaraldehyde (Sigma) for 40 min. The gel was then washed three times in PBS for 20 minutes. Proteins were transferred to nitrocellulose. After blocking, the membrane was incubated overnight at 4° C. with anti-Tβ4 polyclonal antibody (1:1000; Tβ4 1-43, Acris). The membrane was then incubated with horseradish peroxidase-conjugated donkey anti-rabbit immunoglobulin antibody (ImmunoJackson Research) for 1 hour at RT. The specific protein-antibody reaction was detected by the Super signal West Pico Chemoluminescent Substrate (Pierce). Western blots for the evaluation of other proteins were carried out without the glutaraldehyde step and the membranes were incubated with a horseradish peroxidase-conjugated donkey anti-rabbit or anti-mouse immunoglobulin antibody. A monoclonal antibody against phospho ERK (Santa Cruz) was used 1:1,000. Rabbit antibody against total ERK (Cell Signalling) was diluted 1:1,000. Mouse anti-actin antibody (Sigma) was used 1:5000. GluR2/3 rabbit antibody (Chemicon) was diluted 1:1,000. Rabbit anti-N-cadherin (Cell Signalling) 1:1,000. Rabbit anti-β-catenin (Cell Signalling) 1:1,000. The quantitation of protein expression was determined after it was normalised to the respective β-actin by measuring the optical density of respective band blots using the Quantity One software (Biorad).

### Cell Cycle Analysis by FACS

Neurospheres were mechanically dissociated followed by a short passage in diluted trypsin to obtain a cell suspension that was fixed in 2% paraformaldehyde, followed by washes in PBS and suspended in a citrate solution containing propidium iodide (PI; Sigma) and RNAse (Sigma) as described by {Andreassen, 2001 #101}. Cell cycle analysis was performed by FACS (FACS Calibur, Beckton and Dickinson) counting 30,000 events per experiment.

### Electrophysiology

Membrane currents from the cell soma were recorded in the whole-cell configuration of the patch-clamp method {Hamill, 1991 #37} in neural stem cells from 1 to 7 days after plating. Recordings were performed at room temperature using borosilicate glass patch pipettes connected to an Axopatch 200B amplifier (Axon Instruments, Union City, Calif.). The current signal was filtered at 2 KHz, sampled at 10 KHz, and stored on a hard disc. Cells were voltage-clamped at a holding potential of −60 mV. Control and agonist- or antagonist-containing solutions were applied with a gravity-driven system (SF-77B Perfusion Fast Step Warner Instruments, Hamden, Conn., USA). Kainate (Sigma) was dissolved in water and 1-(4-aminophenyl)-3-methylcarbamyl-4-methyl-3,4-dihydro-7,8-methylenedioxy-5H-2,3-benzodiazepine (GYKI 53655) was dissolved in dimethyl sulphoxide before being diluted to their final concentration in standard extracellular bath solution, immediately before use. To record the kainate-induced currents, patch-clamp electrodes were filled with the following solution (in mM): 140 CsCl, 1 EGTA, 10 HEPES-KOH, 6 D-glucose, (pH 7.3). The bath solution was (in mM): 130 NaCl, 3 KCl, 1.5 CaCl₂, 2 MgCl₂, 6 D-glucose, 10 TEA, 10 HEPES/NaOH, (pH 7.3). In Current-clamp recordings, the electrodes were filled with (in mM): 145 K-glu, 1 EGTA, 0.1 CaCl₂, 10 HEPES, 2 MgCl₂, 2 MgATP, 0.3 NaGTP, (pH 7.3). In these experiments, the external contained (in mM): 130 NaCl, 3 KCl, 2 CaCl₂, 10 HEPES, 20 D-glucose, 2 MgCl₂, (pH 7.3).

### Spinal Cord Injury and Transplantation of NPCs

Adult female Swiss (CD1) mice weighing 27-30 g were used (Catholic University Breeding Laboratory, Rome, Italy). The animals were anesthetized with intraperitoneally administered diazepam (2 mg/100 g) followed by intramuscular injections of ketamine (4 mg/100 g). Under aseptic conditions and with the aid of an operative microscope, a T7-T8 laminectomy was performed. A modified aneurysmal clip (80 g/mm²) was then applied for 1 second over the dura mater. Immediately after injury, mice received homotransplants (10⁵ cells in 4 microlitres) of either Empty Vector-NPCs (n=5), Tβ4 antisense-transduced NPCs (n=4) or vehicle medium (n=4) via a glass pipette with a sharp beveled tip 100 □m in diameter which was connected to a Hamilton microdrive syringe. The NPCs or vehicle medium were slowly injected 1-2 mm rostrally into the lesion at 0.2-0.3 microlitre steps over 10 minutes to prevent loss of fluid along the needle tract. After grafting, the skin was closed with metallic clips. During surgery, temperature was monitored and maintained at 37.0±0.5° C. with a heating pad. After surgery, manual bladder expression was performed three times daily until the emptying reflex was established.

### Behavioural Assessment

Gait abnormalities in mice with contusion injury of the spinal cord were assessed weekly by footprint analysis using the CatWalk system (Noldus, Wageningen, The Netherlands) (Hamers et al, 2001, 2006). Briefly, the animals traverse a walkway in a dark room with a glass floor through which light is beamed from the long edge. Light is reflected completely internally. Only when the paw touches the floor light is the light deflected and exits the glass, so that only the contact area is visible. The intensity of the signal depends on the pressure exerted, so that the spot will appear brighter when more weight is put on the paw. Animals crossing the walkway are videotaped using a computer-assisted set-up and digitized data are thresholded in order to extract the paw-floor contact areas and remove background. At least 3 runs per animal were performed in each session. Labels were then assigned to the prints (left and right, fore and hind paws) and several parameters were measured by the Catwalk software, including, a) base of support, i.e. the distance between the central pads of the hindfeet; b) stride length, i.e. the distance between two consecutive prints on each side, c) max area, the maximum area of a paw (in pixels) that comes into contact with the glass plate; d) intensity, the mean brightness of all pixels of the print at max contact (ranging from 0 to 255 arbitrary units). This is a measure of weight support of the different paws; e) print width, the width of the complete paw print; f) print length, the length of the complete print; g) print area, the surface area (in pixels) of the complete print; h) angle of rotation, i.e. the angle formed by the intersection of lines from the left and right prints. Each mouse ran across the CatWalk several times before surgery to establish baseline locomotor parameters. Dotted lines represent the range (mean±s.d.) obtained before surgery.

### Histology

After 8 weeks, grafted animals were deeply anesthetized and transcardially perfused. The spinal cord was dissected, removed, and cryoprotected. Cryostat sagittal sections (400 □m) were used to determine the distribution, profile of transplanted cells and morphometric assessment of tissue sparing/loss. Five sections spanning the region of interest were studied per animal. Alternate sections were stained with either hematoxylin-eosin or cresyl violet for morphological analysis. Brightfield micrographs were obtained with a Leica DMIL microscope connected to a DFC420 camera. Immunostaining using primary antibody directed against □ tubulin III (Chemicon), MAP2 (Sigma-Aldrich), GFAP (Chemicon), nestin (Chemicon), Smi32 (Sternberg Monoclonal Inc.), T□ 4 (N18) (Santa Cruz), GFP (BD Biosciences Clontech), L1 (Santa Cruz), 5-HT (Abcam), and GAP43 (Abcam) was conducted on sections according to standard protocols. Appropriate Cy3/Cy5-conjugated secondary antibodies (Jackson Immunoresearch) were used. In some cases of double immunofluorescence, propidium iodide (PI; Sigma-Aldrich) was used to detect nuclei after RNAse treatment. Sections were counterstained with Hoechst 33342 (Sigma-Aldrich), mounted with an antifading glycerol medium and double/triple labeling of cells was confirmed using confocal microscopy. For quantitation of cell survival, the total number of EGFP-expressing/DAPI-positive cells was counted. Based on our microscopic examination, the size of the cell body of a grafted EGFP-NPC was between 10 and 20 micrometres. We only quantified the EGFP cell bodies that contained a nucleus (identified by DAPI). To quantify the differentiation pattern of transplanted cells, we used confocal microscopy to count the number of EGFP-positive cells that were double-labeled with a different neuronal marker. We then counted the number of EGFP-positive cells that were double-labeled with the neuronal marker in 3 random fields per section. On average, 100-200 cells were counted per field.

### Statistical Analysis

Throughout these Examples, measurements are expressed as mean±SEM and mean±s.d. for behavioral analysis. Statistical analysis was carried out using Student's t test. Data were considered statistically significant if p<0.05.

### Results

Increased N-Cadherin and β-Catenin Expression in Tβ4 Antisense-Transduced Neurospheres which Maintain Unaltered Sphere Morphology and Proliferative Capacity

We first examined the expression of Tβ4 in murine neurospheres by PCR and Western Blot analysis and we found, for the first time, that mouse embryonic NPCs express Tβ4 (FIG. 1 A). To study the effects of the down-regulation of Tβ4 expression, we decided to exploit an antisense strategy mediated by lentiviral infection. Since the lentiviral vector carries the EGFP reporter gene, we were able to sort the cell populations and obtain clones transduced with the empty vector and clones with the Tβ4 antisense vector.

We used Real-Time PCR to verify the presence of the exogenous Tβ4 antisense transcript (FIG. 1B: Antisense Transcript), the expression of the EGFP transcript (FIG. 1B: EGFP Transcript) and the reduction of Tβ4 mRNA level in transduced neurospheres (FIG. 1B: Tβ4 Transcript).

We then selected an antisense clone which showed the highest reduction of Tβ4 mRNA, when compared to either the empty vector clones or the untreated neurospheres. Western Blot analysis on the same clone confirmed a significant reduction of Tβ4 protein due to antisense over-expression (FIG. 1 C).

Next, we focused on the growth and morphology of transduced neurospheres and analysed the expression of progenitor markers. We found that Tβ4 antisense-transduced neurospheres keep a normal morphology with no significant differences in the mean size of the neurospheres (FIG. 2 A) and have similar growth rate (data not shown), as compared with the empty vector-transduced neurospheres. In addition, we assayed the cellular composition of the spheres by the expression of proteins characteristic of immature cells such as nestin {Hockfield, 1985 #38} and glial fibrillary acidic protein (GFAP) {Bignami, 1972 #39}. By triple immunofluorescence, we showed that all cells in the spheres express nestin, some co-express GFAP. No significant differences between the two clones were observed (FIG. 2 B). Furthermore, we did not detect expression of the neuronal marker microtubule-associated protein 2 (MAP2), as demonstrated by Western blot analysis (FIG. 2 C).

The over-expression of Tβ4 in colon carcinoma cells can cause down-regulation of E-cadherin {Wang, 2004 #40}, resulting from the disruption of the adherence junction due to the depolymerisation of actin microfilaments triggered by this peptide {Huang, 2006 #41}. Hence, we asked whether the down-regulation of Tβ4 expression in neurospheres could induce changes in N-cadherin expression, which mediate calcium-dependent adhesion in the central nervous system. Indeed, Western blot analysis revealed a 5-fold increase in the expression of N-cadherin in Tβ4 antisense-transduced neurospheres (FIG. 2 C). In parallel, we also found an increased expression of β-catenin which is normally complexed with N-cadherin. Densitometric analysis showed a 4.6-fold increase of β-catenin in Tβ4 antisense extracts as compared to control (FIG. 2 C).

Because the process of mitosis is highly dependent upon actin dynamics {Glotzer, 2005 #42}, we asked whether down-regulation of Tβ4 could affect cellular division and, more specifically, the phase of cytokinesis during which a ring of actin has to be formed to allow physical cell division. To this aim, we performed a cell cycle analysis by FACS of the randomly cycling transduced neurospheres. We did not detect any accumulation of aneuploid cells in Tβ4 antisense-transduced neurospheres, which showed a cell cycle profile similar to the empty vector cells, even after several passages in culture (FIG. 3 A upper panel). Indeed, we also found a comparable distribution of cells in the different phases of cell cycle between the two transduced clones (FIG. 3 A lower panel). Two transduced mitotic cells in telophase are shown (FIG. 3 B).

In conclusion, the antisense strategy was efficient to reduce Tβ4 expression levels in mouse embryonic NPCs without affecting the growth, morphology or the expression of markers generally expressed in undifferentiated neurospheres. However, Tβ4 down-regulation in NPCs significantly elevated the expression of N-cadherin and β-catenin.

### Down-Regulation of Tβ4 Influences Various Aspects of Neuronal Differentiation of NPCs

During differentiation, neurons extend membrane protrusions some of which develop into neurites, characterised by a guiding growth cone and branch formation. These morphological changes largely depend on the dynamics of the actin cytoskeleton which is regulated by a variety of actin-binding proteins {Stossel, 1989 #43} {Tanaka, 1995 #44} {da Silva, 2002 #45}. Tβs are abundant in neural tissues where they appear to have a role in neurite development {van Kesteren, 2006 #31} {Carpintero, 1999 #46} {Choe, 2005 #47}. Although progress has been made on understanding the function of Tβ4 in the nervous system, many aspects still need to be further investigated. Here, we used NPCs to elucidate Tβ4 role during neuronal differentiation and neuritogenesis.

After withdrawal of EGF and bFGF, neurospheres adhere, break their spherical structure and cells differentiate and acquire the morphological properties of neurons and glia {Reynolds, 1992 #50} {Gage, 1995 #48} {Vescovi, 1993 #49}. For over a week we followed the differentiation of NPCs into neurons by the expression of specific markers such as β tubulin III and MAP2. Generally, we noted that, in differentiating conditions, nestin-positive cells decreased (data not shown), whereas the percentage of glia (GFAP positive cells) and neurons increased. No cell was simultaneously positive for GFAP and β tubulin III. The β tubulin III positive cells had a characteristic neuronal phenotype, with a small soma, two or few neurites, and laid above a layer of larger GFAP-positive cells (FIG. 4 A).

First, to analyse the subcellular localisation of Tβ4 in differentiating neurons derived from mouse NPCs, we performed immunofluorescence staining. Confocal images of developing neurons, β tubulin III positive, showed a Tβ4 staining which was distributed in the cell body, growth cone and distal tips of neurites (FIG. 4 B). Double immunofluorescence with synaptophysin, a synaptic vesicle marker, showed a colocalisation pattern of the two antigens mostly along processes (FIG. 4 C). The specific localisation in growth cones and processes, in agreement with previous observations {Choe, 2005 #47} {Moccia, 2003 #73} {van Kesteren, 2006 #31}, indicates that Tβ4 is involved in neurite outgrowth of NPCs under differentiating conditions. Next, we measured by Real-Time PCR the RNA level of Tβ4 in NPCs cultured in differentiating conditions and found that they have a significantly lower level as compared to neurospheres, thus indicating that a down-regulation of Tβ4 usually occurs during differentiation. This decrease was, as expected, higher in cells transduced with Tβ4 antisense (FIG. 4 D). Indeed, Tβ4 protein levels were undetectable by Western Blot analysis in differentiated cultures (data not shown). We followed over 7 days the differentiation of neurons derived from transduced NPCs, by the expression of either β tubulin III or the dendritic marker MAP2. Triple labelled confocal images showed that neurons from the empty vector-transduced clone were present in lower number when compared with similar fields of Tβ4 antisense-transduced cultures (FIG. 5 A). This difference in neuronal number was confirmed by labelling with both neuronal markers. In particular, neurons derived from the empty vector-transduced clone, represented on average the 5% of the cell population in culture, whereas the cell population derived from the Tβ4 antisense-transduced neurospheres, showed a two-fold increase in the number of cells positive for neuronal markers. Indeed, Western blot demonstrated a 2-fold increase of MAP2 in differentiating cultures transduced with Tβ4 antisense compared to the empty vector, as revealed by densitometric analysis (FIG. 5 B).

Tβ4-antisense neurons were morphologically different from neurons derived from the empty vector-transduced clone. As shown by immunofluorescence (FIG. 5, C and D), starting from the early phases of differentiation, the overall Tβ4 antisense differentiating neurons rapidly extended processes and had more prominent neurites. Interestingly, as shown in FIG. 6 A, Tβ4 antisense-transduced neurons established a net of connections, and had an increased number of neuronal processes emanating from the cell body (see also FIGS. 5 A and D), thus acquiring a multipolar aspect. In addition, neurites of Tβ4 antisense-transduced neurons were about twice as long as those of empty vector-transduced neurons (FIG. 6 B), and this difference was observed up to day 7 (FIG. 6 C). All together these results indicate that Tβ4 has a role in neuronal fate decision of NPCs and possess an outgrowth-promoting activity.

Down-Regulation of Tβ4 in Developing Neurons is Associated with the Increased Expression of N-Cadherin/β Catenin and ERK Activation

The above data showed that the down-regulation of Tβ4 is able to influence neuronal fate decision, and neurite elongation. Previous studies reported that N-cadherin is essential for differentiation and nerve cell morphology, and that N-cadherin over-expression is sufficient to initiate neuronal differentiation in P19 and PC12 cells {Utton, 2001 #51} {Gao, 2001 #52} {Doherty, 2000 #53} {Chen, 2005 #54}. We therefore asked whether Tβ4 down-regulation could increase N-cadherin expression in differentiating cultures. Western blot analysis on protein extracts of cultures after 3 days of differentiation, showed a 1.6-fold increase of N-cadherin in Tβ4 antisense compared to the empty vector (FIG. 7, right, upper panel). In addition, as indicated by densitometric analysis, β-catenin levels were also significantly increased (1.8 fold) (FIG. 7, right, middle panel).

Mammalian ERK1 (p44) and ERK2 (p42) are the best characterised members of the MAP (mitogen-activated protein) kinase family and are activated by concurrent phosphorylation of threonine and tyrosine residues {Blenis, 1993 #55} {Cano, 1995 #56}. Several data indicate that ERKs activation is required for full neurite outgrowth induced by N-cadherin {Perron, 1999 #57} {Saffell, 1997 #58} {Utsugisawa, 2002 #59}. Therefore, in the next step, we analysed ERK phosphorylation levels in differentiating culture extracts. Although, the levels of total ERKs was increased by approx. 20%, quantification of the immunoreactive levels of the activated kinases, normalised by the total amount of the respective kinase, revealed a 2-fold increase in the phosphorylation state of ERK1 in Tβ4 antisense extracts (FIG. 7, right, lower panel).

These biochemical data suggest that Tβ4 down-regulation may influence neuronal differentiation of NPCs probably by increasing the expression of the adhesion complex N-cadherin/β-catenin and ERK1 activation.

### Down-Regulation of Tβ4 Increases AMPA Receptors in Tβ4 Antisense-Transduced Neurons

As next step, we further characterised the phenotype of the transduced neurons by electrophysiological experiments. To confirm neuronal identity, single-cell Real-Time PCR was performed on recorded cells to detect the expression of the neuronal marker MAP2 (data not shown). A comparative analysis showed that resting membrane potential for the two neuronal populations was not significantly different. In fact, at day 1 the mean resting potential of control neurons (n=6) was −27.66±1.15 mV, and for Tβ4 antisense-transduced clone, was −29.12±4.48 mV (n=8). Similarly, the resting potential at day 7 was −39.47±1.71 mV for control neurons (n=19) and −36.11±3.05 for Tβ4 antisense-transduced neurons (n=22). FIG. 8 A shows an example of developing neuron used for the whole-cell patch-clamp recording.

Tβ4 antisense neurons and control neurons showed action potential evoked by 50 pA of current injection from day 2. However, all neurons did not show repetitive firing but just one or two action potentials (FIG. 8 A). In voltage-clamp mode, depolarising steps from holding potential of −60 mV (from −50 mV to +40 mV, step 10 mV), evoked currents with an early inward peak, the voltage-dependent sodium currents, followed by outward components, the voltage-dependent potassium currents (FIG. 8 B). In the insert is shown a magnification of the early component that was blocked by 1 μM of tetrodotoxin (TTX) (not shown), thus indicating that these early currents were due to voltage-activated sodium channels. No statistical differences were observed in the amplitude of both inward and outward currents.

Subsequently, we tested the cellular response to kainate injection. All patched neurons responded to perfusion of kainite (200 μM) with a current that was blocked by GYKI 53655 (100 μM), a selective AMPA receptor antagonist (FIG. 8 C), confirming that the activation of these receptors was responsible for the current. In control neurons, the amplitude of the kainate-induced currents increased with time in culture and, at day 7, the mean current was 110±15 pA (n=19). Tβ4 antisense neurons responded to kainate injection with a current that was significantly higher compared to control neurons starting from day 1 (FIG. 8 E). At day 7 the amplitude of the currents for control and Tβ4 antisense neurons was, instead, not significantly different (FIG. 8 E). Such an increase in current density could result from the insertion of more channel receptors at the plasma membrane, or from changes in the intrinsic properties of the channels. Western blot analysis on differentiated cells at day 3 showed a 2-fold increase of GluR2/3 subunits in Tβ4 antisense neurons (FIG. 8 F), thus suggesting an increased AMPA receptor surface expression. Based on the timing and higher number of glutamate-AMPA receptors, we concluded that Tβ4 down-regulation induces a more rapid differentiation of NPCs towards a neuronal phenotype.

Animals Grafted with Tβ4 Antisense-Transduced NPCs Show Improved Locomotory Function in a Mouse Spinal Cord Injury Model

Based on the in vitro results showing increased differentiation of Tβ4 antisense-transduced NPCs, we investigated the effects of their transplantation on locomotory recovery in a mouse model of spinal cord injury. In this model of spinal cord contusion injury, a complete palsy of the hindlimbs lasts for 2 weeks, followed by a stepping gait with partial recovery of locomotory activity over the subsequent 6-8 weeks (Pallini et al., 1988; Pallini et al., 1989).

NPCs, genetically modified to express the EV or Tβ4 as, were transplanted as small neurosphere suspensions just rostrally to the injury site. Dissociation to generate single cell suspension was avoided to exclude detrimental effects on cell survival. At the time of transplantation, cells expressed nestin and fewer than 1% expressed the astroglial marker GFAP.

To determine whether transplantation of NPCs improved recovery of function in mice bearing spinal cord contusion injury, we performed footprint analysis (FIG. 9A upper panel) using the automated CatWalk system that monitors different gait parameters. Compared with observational open field methods, which monitor the animals' reluctance to move about in an open field arena and that have been used in similar studies, the footprint analysis provides a more reliable assessment of hindlimb movements. Mice transplanted with either EV-(n=5) or Tβ4 antisense-transduced NPCs (n=4), recovered a stepping gait one week after spinal cord injury, while animals that received vehicle injection (n=5) experienced this recovery after 2 weeks (FIG. 9A lower panel). Analysis of gait parameters showed that the stride length and intensity recovered significantly better in mice grafted with Tβ4 antisense-transduced NPCs than in mice grafted with Empty Vector-NPCs.

Stride length (R&L) of mice grafted with Tβ4 antisense-transduced NPCs at 4 and 8 weeks after injury was 53.3±1.8 and 56.9±2.2 mm respectively, compared with the stride length (R&L) of mice grafted with Empty Vector-NPCs, which was 48.5±3.4 and 53.9±1.9 mm (p=0.004 and p=0.023; mean±s.d., Student's t-test) (FIG. 9A lower panel and B). The gait intensity of mice grafted with Tβ4 antisense-transduced NPCs at 4 and 8 weeks after injury was 31.9±5.8 and 41.9±7.7 respectively, while that of mice grafted with Empty Vector-NPCs was 20.3±6.9 and 29±1.8; (p=0.032 and p=0.009; mean±s.d., Student's t-test) (FIG. 9B). There were no significant differences in the two groups of animals in the other parameters measured, including the base of support, maximum area, print width, print length, print area and angle of rotation (FIG. 9B).

Overall, this analysis revealed a better outcome in injured mice grafted with Tβ4 antisense-transduced NPCs.

### Transplanted Tβ4 Antisense-Transduced NPCs Survive and Differentiate in Injured Mouse Spinal Cord

To investigate the morphological basis and cellular mechanisms which may contribute to the motor recovery, we analyzed sagittal serial sections from mice grafted with Tβ4 antisense-transduced NPCs and Empty Vector-NPCs, 8 weeks after spinal cord contusion. The gross morphology of the injured spinal cord was visualized by hematoxylin-eosin staining, which identified the damaged area marked by the accumulation of a cellular and connective tissue scar (FIG. 10A). The quantification of the lesion area in consecutive sections showed that the lesion remains unchanged in the two groups of grafted/injured mice (data not shown). We then performed double/triple immunofluorescence labeling studies to identify the survival, localization and differentiation of transplanted NPCs in the two groups of injured mice.

Grafted NPCs were detected by direct examination of the EGFP fluorescence in the injured spinal cords. Examination of sagittal sections 8 weeks after injury in both groups of mice identified nuclei positive for EGFP surrounding and within the lesion site, suggesting that following transplantation many of the cells were attracted towards the lesion site. FIGS. 10B and C show, by triple-staining, examples of the distribution of EGFP-positive cells in sagittal sections from animals receiving transplants of the Empty Vector- or Tβ4 antisense-transduced—NPCs. In the images, survival and localization of the EGFP-positive cells were visualized along with the nuclei and GFAP-positive cells which, as previously reported (Camand et al., 2004), form a glial scar consisting mainly of reactive astrocytes surrounding the center of the lesion. The survival in the injured area of Tβ4 antisense-transduced—NPCs was notably higher than that of Empty Vector-NPCs (FIGS. 10B and C), which in two mice were barely detectable and appeared unhealthy as judged by the appearance of their fluorescence. To confirm the presence of transplanted cells and to rule out auto-fluorescent cell debris, we immunostained a selection of transplanted spinal cord sections also with an anti-GFP antibody. FIG. 10D shows that the EGFP-positive cells are colabeled with the anti-GFP antibody and confirms that they were more abundant in mice which received transplants of T□4 antisense-transduced—NPCs.

We also confirmed that the Tβ4 antisense-transduced NPCs maintain a down-regulation of the peptide in vivo by performing immunofluorescence with an antibody specific for Tβ4. As can be seen in FIG. 10E, transplanted T□4 antisense-transduced-NPCs displayed only the green fluorescence, while transplanted Empty Vector-NPCs showed colabeling of the EGFP with Tβ4 staining.

To investigate the phenotype of the cells derived from the grafted NPCs, spinal cord sections were immunostained with anti-GFAP antibody for astrocytes and anti-β tubulin III or MAP2 or Smi32 antibody to stage neuronal differentiation. It is interesting that, while NPCs expressed nestin at the time of transplantation, nestin immunofluorescence was no longer detectable 8 weeks after the grafts (data not shown), indicating that NPCs entered a differentiation program in vivo. In contrast, as confirmed by earlier reports (Frisen et al., 1995; Namiki and Tator, 1999; Shibuya et al., 2002; Sieber-Blum et al., 2006), intense nestin fluorescence was present in the host tissue near the lesion site (data not shown).

In injured mice transplanted with the Empty Vector-NPCs, EGFP-positive cells showed a rounded shape, never displayed a neuronal-like morphology and did not express neuronal antigens (FIGS. 11A and B). The cellular morphology of the Tβ4 antisense-transduced—NPCs engrafted into injured spinal cord showed that a large proportion of these cells remained undifferentiated, maintaining a rounded shape and forming clusters (FIGS. 10C and D). Interestingly, some Tβ4 antisense-transduced NPCs exhibited multipolar extended processes resembling neuronal cells and were generally found in the area surrounding the lesion (FIG. 11A). To verify their neuronal differentiation, we then performed immunofluorescence with specific markers. This analysis showed that while grafted Empty Vector-NPCs remain rounded and undifferentiated, a small number of the Tβ4 antisense-transduced NPCs, ranging from 0.1 to 0.2%, displayed a neuronal-like morphology and expressed either β-tubulin III, MAP2, or Smi 32 (FIG. 11B).

These data suggest that a low percentage of Tβ4 antisense-transduced NPCs are capable of terminal differentiation along a neuronal lineage in an inhospitable environment, such as the injured spinal cord.

### Transplanted Tβ4 Antisense-Transduced NPCs Over-Express L1 and Promote Regeneration and Sprouting of Host Fibers in Injured Mouse Spinal Cord

The small number of Tβ4 antisense transplanted cells differentiatied into neurons was insufficient to account for the functional recovery observed in grafted/injured mice. We therefore explored the hypothesis that engrafted T□4 antisense-transduced NPCs may provide an environment conducive to the attachment and growth of endogenous neural and neuronal cells. Tβ4 plays a pivotal role in regulation of actin dynamics in neurons and is involved in cell survival and neurite elongation by influencing cytoskeleton changes and the redistribution of cell adhesion molecules (Yang et al., 2008). In particular, it has been proposed that Tβ4 exerts its neuropromoting effects at least partly via mechanisms that involve the activation of cell adhesion molecule L1.

The neuronal recognition molecule L1 has been shown to favour axonal growth in an inhibitory environment (Castellani et al., 2002; Chen et al., 2005a; Chen et al., 2007; Roonprapunt et al., 2003; Xu et al., 2004; Zhang et al., 2005). Based on these observations, we investigated whether the functional improvement of mice transplanted with Tβ4 antisense-transduced NPCs might be due to increased L1 expression. In FIG. 11C, an analysis of a series of confocal images shows that Tβ4 antisense-grafted NPCs over-express L1 and create an environment that is conducive to host neurite regrowth. Numerous anti-β tubulin III-positive processes were found to extend into areas rich in transplanted Tβ4 antisense-transduced NPCs. On close inspection, it appeared that host neurites were attracted to and made contact with Tβ4 antisense-grafted NPCs in the lesioned area (FIG. 12A arrows), most likely due to the ability of these cells to express L1.

Furthermore, as shown by double immunofluorescence (FIG. 12B), β tubulin III-positive fibers were also immunostained for GAP43, which specifically marks enhanced axon growth status and axonal regeneration (Schreyer and Skene, 1993). This finding shows that the microenvironment after transplantation of the Tβ4 antisense-transduced NPCs is more hospitable to axon regeneration.

Given the fact that serotonin (5-HT) fibres play important roles in locomotion (Barbeau and Rossignol, 1991) and in recovery after injury (Ribotta et al., 2000), we examined whether Tβ4 antisense-transduced NPCs over-expressing L1 promote sprouting of 5-HT axons. By immunofluorescence, we found a high density of 5-HT fibres localized around Tβ4 antisense-transduced NPCs in the proximity of the lesion site (FIG. 12C). Since 5-HT fibres provide a diffuse innervation and positively correlate with a degree of functional recovery (Pearse et al., 2004; Ribotta et al., 2000), we suggest that a more vigorous regeneration and sprouting of host fibres may explain the enhanced improvement in gait.

### FIGURE LEGENDS

FIG. 1: Down-regulation of Tβ4 expression in mouse embryonic neurospheres

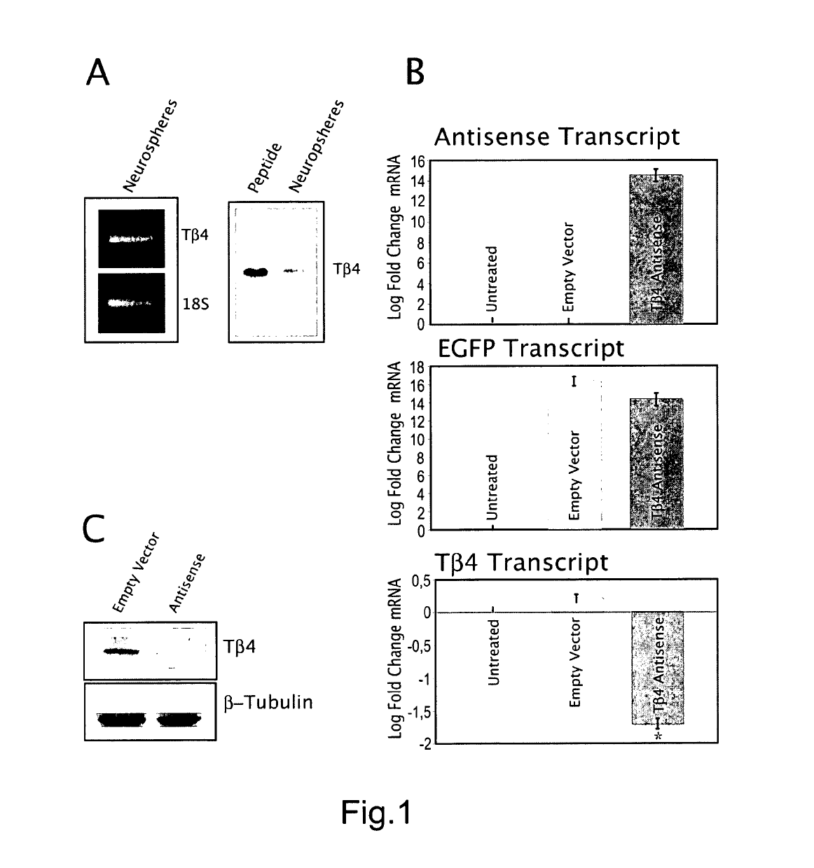

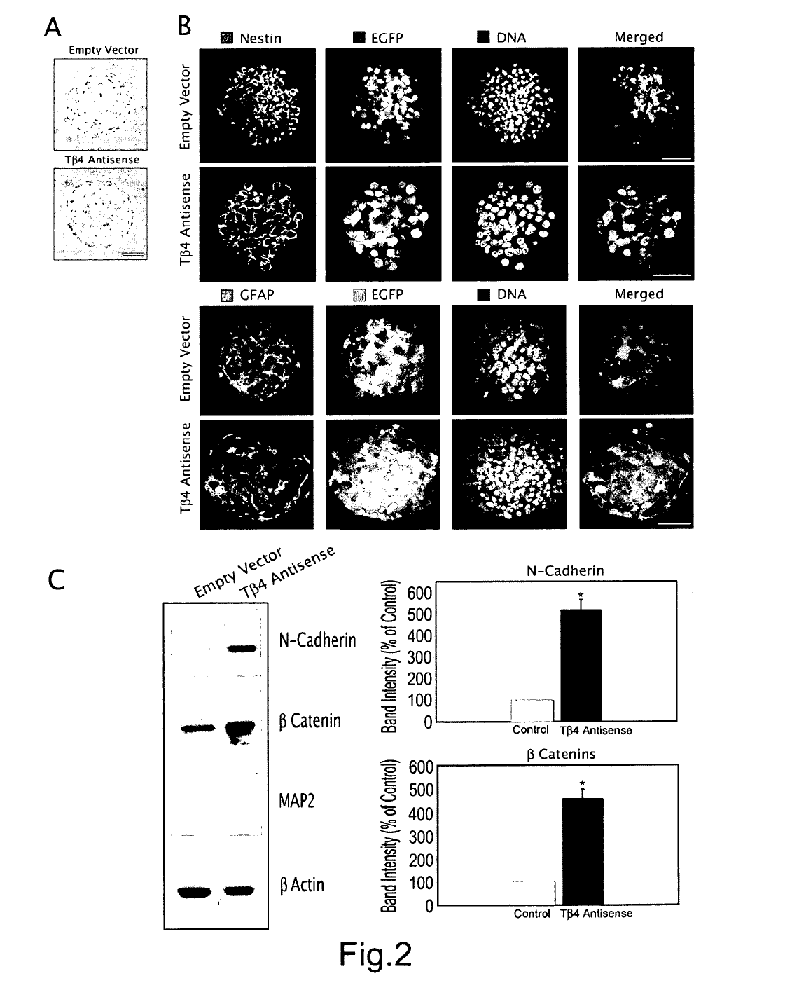

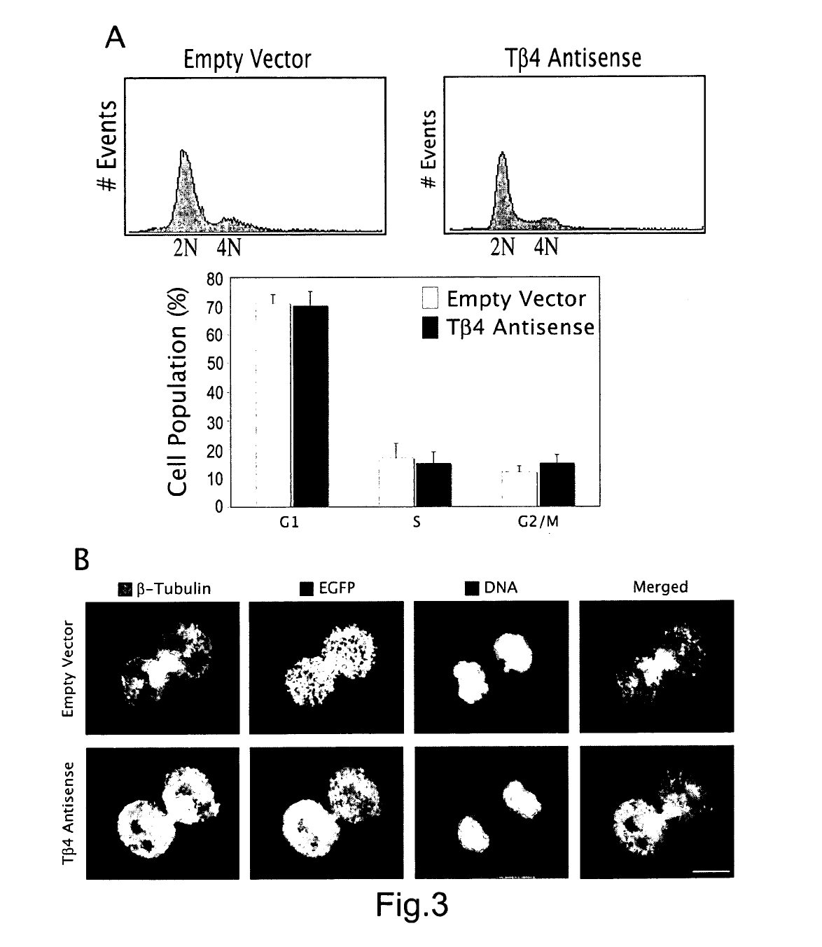

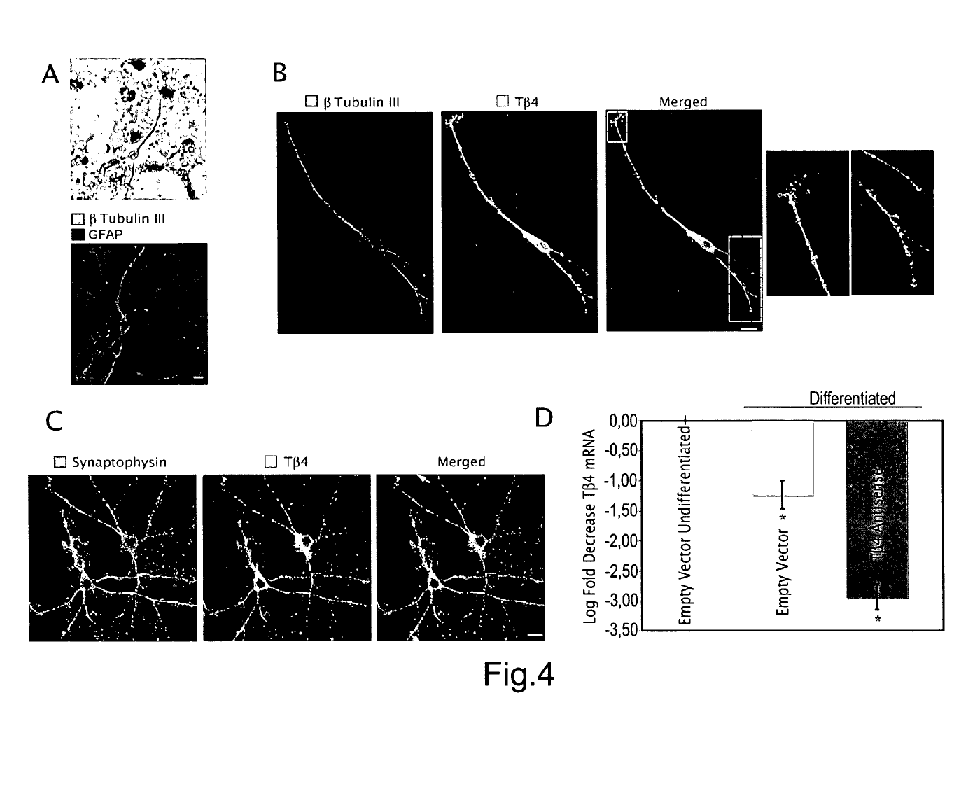

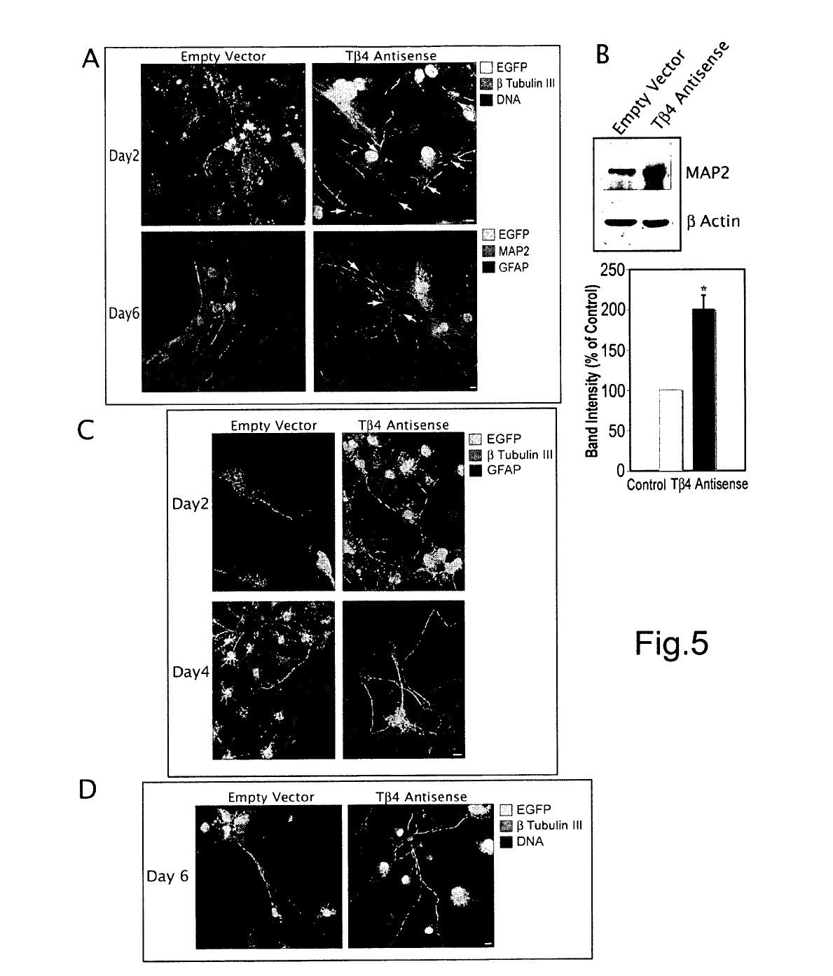

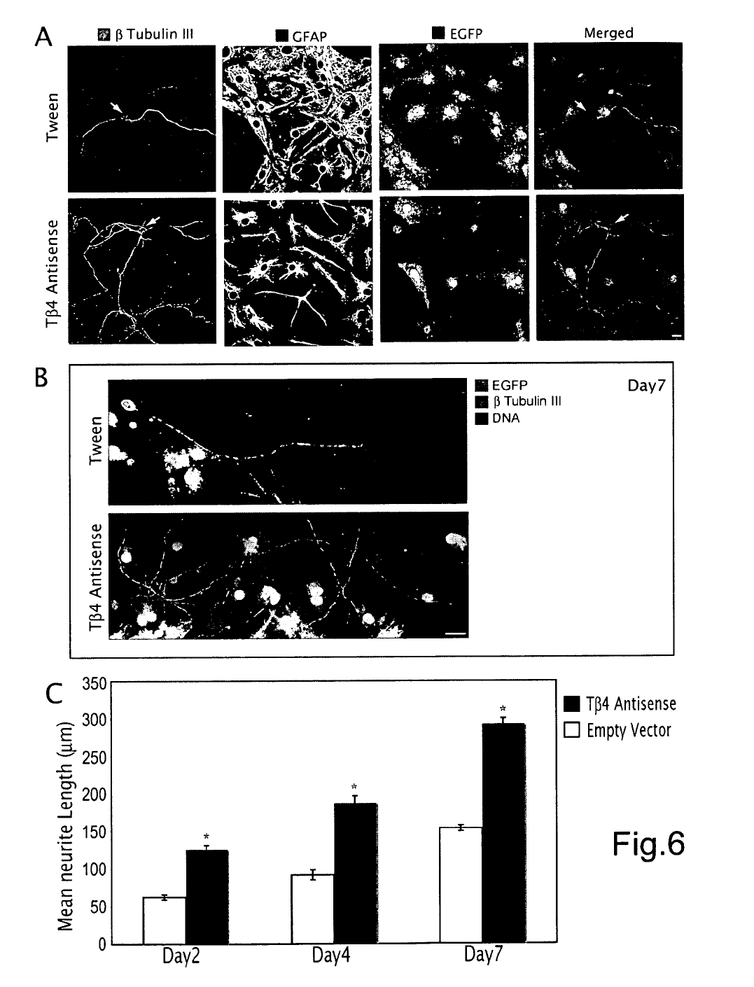

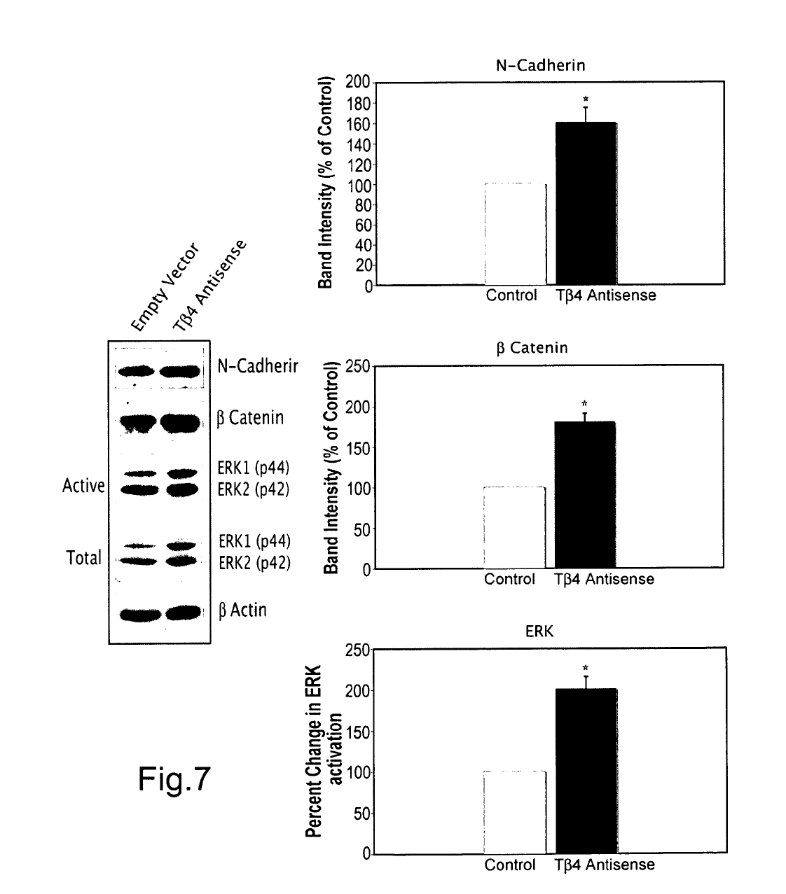

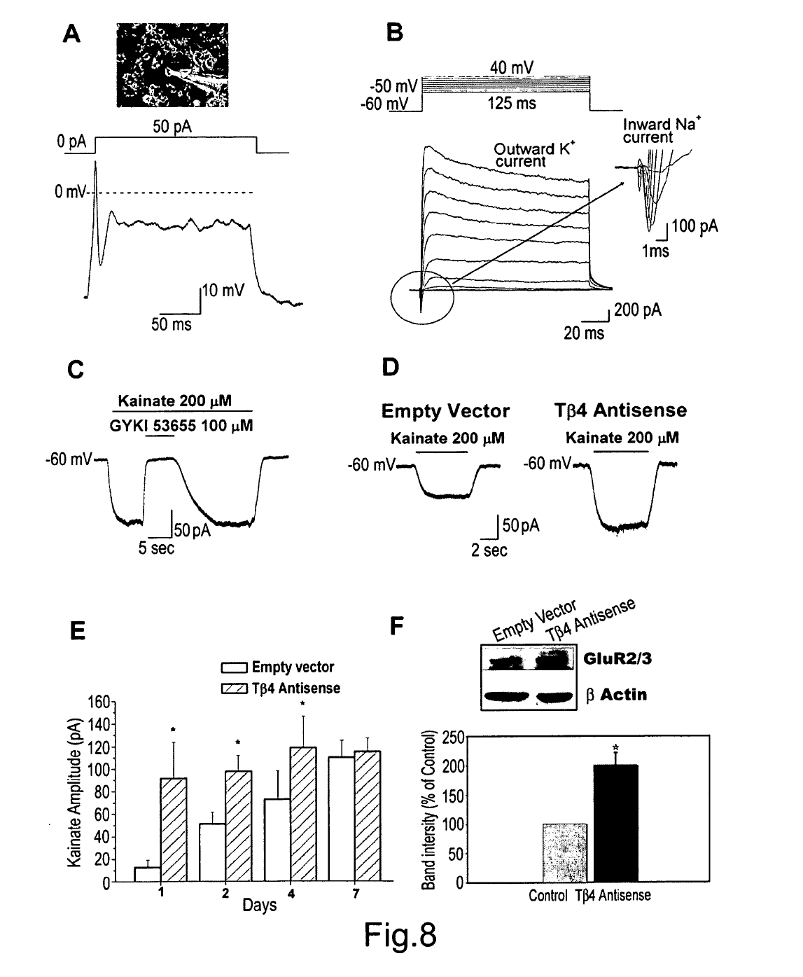

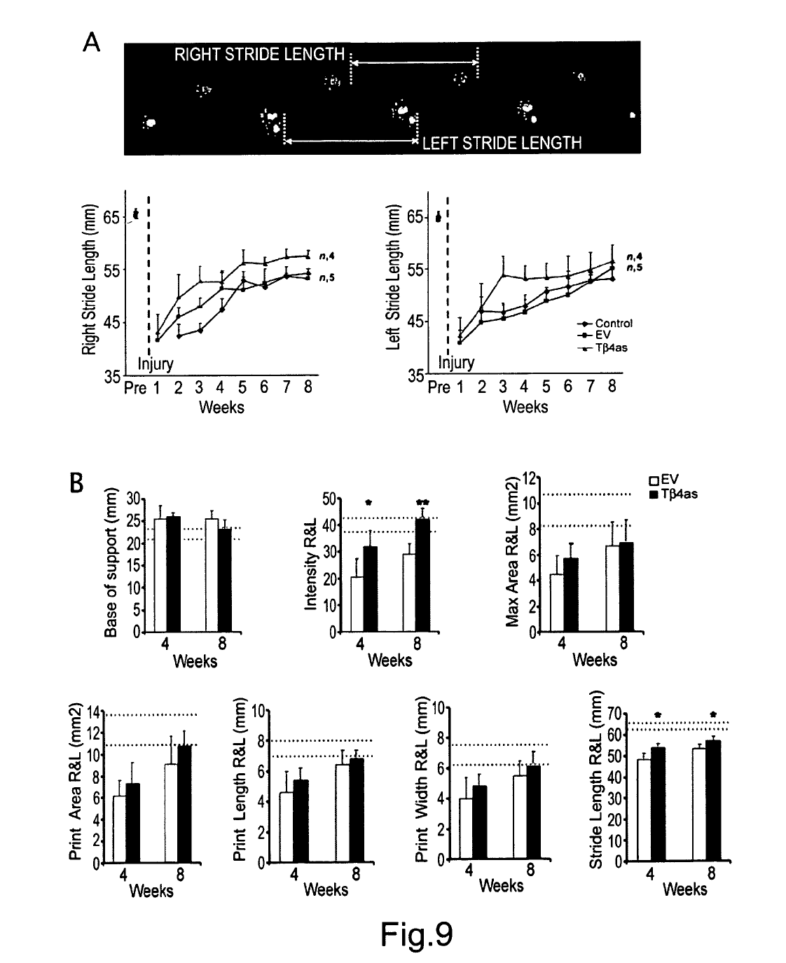

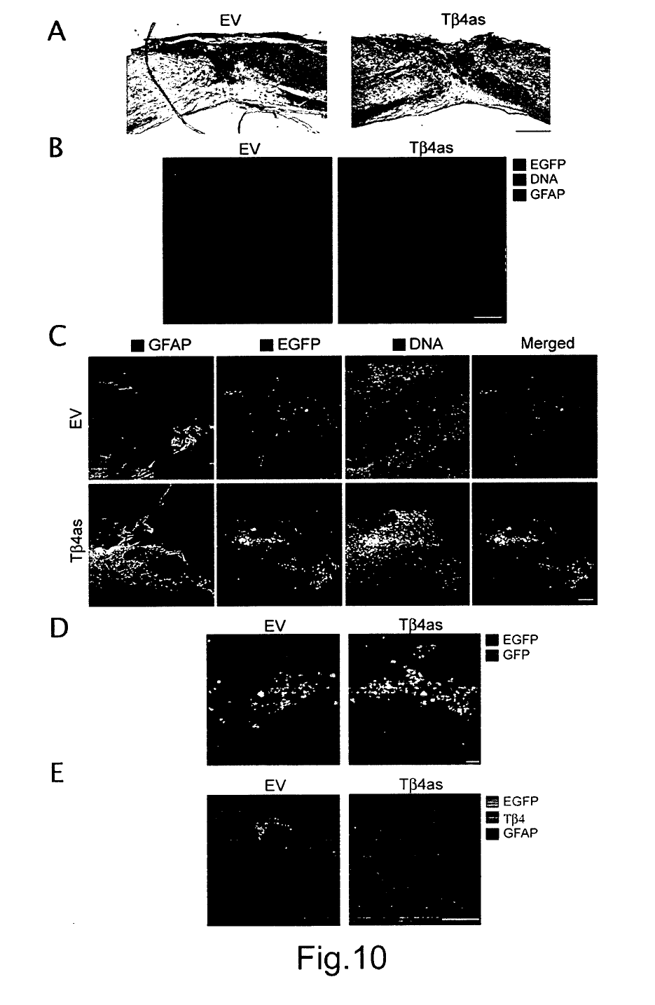

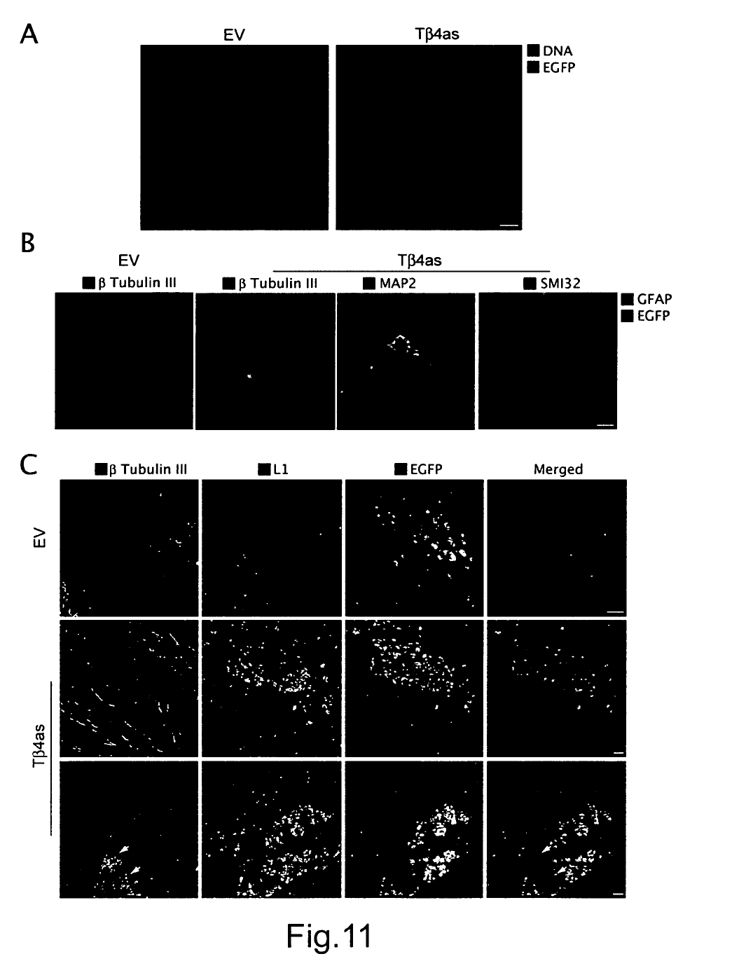

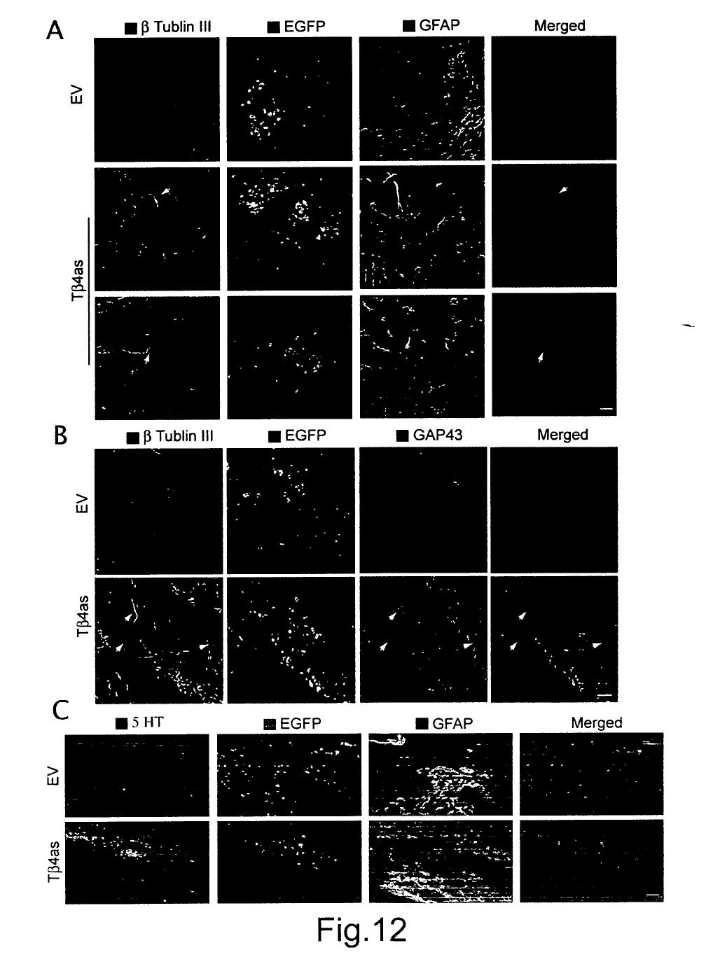

A. Left panel. RT-PCR analysis of Tβ4 transcript expression in neurospheres cultured in undifferentiated conditions. A single band of the expected size (120 bp) was obtained using oligonucleotides annealing to the mouse coding sequence. Amplification of 18S rRNA (151 bp) was carried out in parallel. A. Right panel. Western blot analysis of Tβ4 peptide expression in extracts of undifferentiated neurospheres (passage 5). A single band at approx. 5 kDa is detected by the anti-Tβ4 polyclonal antibody which migrates as the purified Tβ4 peptide.

B. Real-Time PCR was performed on total RNA of untreated and lentiviral transduced neurospheres. In undifferentiated neurospheres, the relative expression of the following was evaluated: exogenous Tβ4 antisense transcript, distinguishable for the presence of the HA-tag sequence; EGFP transcript; and total Tβ4 transcript. In the Tβ4 antisense-transduced neurospheres there is a strong over-expression of the exogenous transcript, which is undetectable in both untreated and empty vector neurospheres. On the contrary, Tβ4 transcript expression is significantly reduced only in Tβ4 antisense-transduced neurospheres as compared to both untreated and empty vector-transduced neurospheres. The expression of EGFP transcript is similar in Tβ4-antisense and empty vector neurospheres, confirming that the effect of down-regulation is specific for the Tβ4 transcript. Values are plotted as log (base2)-fold change of calibrator (empty vector sample). 18S rRNA expression was used for each sample normalisation. *p<0.01 vs. control values.

C. Western blot analysis performed on undifferentiated transduced neurosphere confirms a reduction of Tβ4 protein level as compared to empty vector extracts. Detection of β-tubulin was used to confirm equal protein loading.

FIG. 2; Tβ4 antisense-transduced mouse embryonic neurospheres have unaltered morphology and expression undifferentiated markers but over-express N-cadherin and β-catenin

A. Phase contrast images of undifferentiated transduced neurospheres. No major morphological differences were detected between the two groups. B. Upper panel: transduced neurospheres labelled with an anti-nestin antibody (red) and DNA (blue). Lower panel: transduced neurospheres stained with an anti-GFAP antibody (red) and DNA (blue). The EGFP labelling (green) is always present because it is carried by the lentiviral vector used for cellular transduction. All cells in the transduced neurospheres express nestin, whereas GFAP is expressed only in some cells of the spheres. Bars, 50 μm.

C. A representative panel of Western blots carried out on undifferentiated transduced neurospheres is shown. Tβ4 antisense-transduced neurosphere extracts show an increased expression of N-cadherin and β-catenin but do not express the dendritic marker MAP2. β-actin was used as a loading control. Densitometric analysis was carried out using the Quantity One software (Biorad) and the normalised amount of each protein is shown in the graphs. Bars in the plot represent means±SEM. * p<0.01 vs. control values.

FIG. 3: Tβ4 antisense-transduced NPCs do not show altered proliferative capacity

A. Upper panel: flow cytometric analysis of DNA content in randomly cycling undifferentiated neural cells. Empty vector and Tβ4 antisense-transduced NPCs have similar cell cycle profiles, as shown by representative histograms of DNA content. Lower panel: the graph shows similar percentages of cell population in G1, S and G2/M phases of the cell cycle in the two transduced neurosphere clones. Cell cycle distributions displayed are representative of three experiments. B. Confocal images showing two isolated transduced NPCs in telophase fixed and stained for β tubulin (red) and DNA (blue). Both cells are positive for the EGFP (green) due to the presence of the lentiviral vector. No major differences are observed in the late phase of mitosis between Tβ4 antisense-transduced NPCs and control cells. Bar, 10 μm.

FIG. 4: Tβ4 protein is localised in neuronal processes and growth cones of developing neurons derived from NPCs

A. Upper panel: a phase contrast image illustrating a neuron (white arrow), developing above a layer of flat and larger glial cells. Lower panel: a confocal image showing a neuron, positive for β tubulin III (red), extending with its processes over a layer of glial cells positive for GFAP (blue). Bar, 10 μm.

B. Double immunofluorescence staining with β tubulin III (red) and Tβ4 (green) of a 3 day differentiating neuron. Tβ4 labelling is present in cell body, neurites and growth cone (white square boxes). Enlargements of the areas comprised within white boxes, show that Tβ4 labelling is found in the growth cone, in varicosities along the process and at process tips. C. Double immunofluorescence staining with synaptophysin (red) and Tβ4 (green) of a day 8 differentiating neuron. The staining for both antigens is mostly overlapping, has a somewhat granular appearance, and is detected in the cell body and along processes. Arrows indicate Tβ4 staining-enriched at the distal portions of neurites. Bar, 10 μm. D. Real-Time PCR shows that NPCs cultured in differentiating conditions have a significantly lower mRNA level of Tβ4 as compared to neurospheres. * p<0.01 vs. control values. The decrease is higher in cells transduced with Tβ4 antisense. * p<0.01 vs. empty vector.

FIG. 5: Tβ4 antisense-transduced NPCs generate a higher number of neurons with an increased neurite extensions

A. Upper panel: confocal images of differentiating transduced NPCs, labelled with β tubulin III (red), DNA (blue). Lower panel: confocal images of differentiating transduced NPCs, labelled with MAP2 (red) and GFAP (blue). The EGFP is common to all images since it is carried in the lentiviral vector. In comparable fields, at day 2 as well as day 6 of differentiation, neurons (white arrows) are more numerous in Tβ4-antisense cultures as compared to empty vector cultures. Note that Tβ4-antisense neurons generate a more complex net of connections. B. Western blotting confirms that Tβ4-antisense extracts demonstrate a two-fold increase of the dendritic marker MAP2 as shown by densitometric analysis. Bars in the plot represent means±SEM. * p<0.01 vs. control values.

C. More examples of differentiating transduced NPCs at different days of differentiation, labelled with β tubulin III (red) and GFAP (blue). Tβ4-antisense cultures, from the early days of differentiation, show neurons with more prominent and longer neurites. D. A field of transduced NPCs, at day 6 of differentiation, labelled with β tubulin III (red) and DNA (blue). Generally, empty vector neurons have a bipolar morphology whereas Tβ4-antisense neurons show longer and more prominent neurites, and a higher number of processes emanating from the cell body, giving them a multipolar morphology. Bars, 10 μm.

FIG. 6: Neurons derived from Tβ4 antisense-transduced NPCs develop, from the early days of differentiation, neurites twice as long as those of control neurons

A. Confocal images of differentiating NPCs labelled with β tubulin III (red), GFAP (blue) and positive for EGFP (green). Each channel has been separated from the merged image to better distinguish the different labelled cells. Representative fields of neurons derived from the empty vector clone and Tβ4-antisense clone, are shown. The empty vector, β tubulin III-positive neuron (red) has a bipolar morphology, whereas the Tβ4-antisense β tubulin III-positive neuron (red) shows a higher number of prominent processes departing from the cell body (white arrows). B. Immunofluorescence images reconstructed by joining two contiguous cellular fields shown that neurites from a Tβ4-antisense neuron are twice as long as those of a control neuron, identified by the β tubulin III staining (red). D. The histogram shows the quantitation of the total neurite length over 7 days of differentiation. Tβ4-antisense neurons show significantly enhanced outgrowth of neurites compared with control neurites (n=20) Bars in the plot represent means±SEM. *p<0.01.

FIG. 7: Tβ4 down-regulation in differentiating cultures induces over-expression of N-cadherin/β-catenin and activates ERK1

Left panel: extracts from differentiating cultures were analysed by Western blotting for N-cadherin, β-catenin, total ERKs, active ERKs and β-actin. Right panel: densitometric analysis of the Western blot bands for N-cadherin and β-catenin, both normalised to β-actin, shows respectively a 1.7 and a 1.8-fold increase in Tβ4 antisense extracts as compared to control (empty vector). Quantitation of immunoreactive levels of activated and total ERKs, normalised by the total amount of the respective kinase, revealed a 2-fold increase in the phosphorylation state of ERK1 in Tβ4 antisense extracts. Bars in the plot represent means±SEM. *p<0.01.

FIG. 8: Tβ4 antisense-transduced neurons have distinct electrophysiological properties

A. Phase-contrast micrograph of a differentiating field of NPCs. A cell during patch clamp recording is shown. Lower panel: a representative response evoked by 50 pA of current injection is shown. The cell was at day 2 of differentiation and did not show repetitive firing. B. Currents evoked by 125 ms depolarising steps from a holding potential of −60 mV. Depolarising steps were delivered every 3 seconds. In this Figure it is possible to see the transient potassium current component, represented by the peak of the traces, and the delayed rectifier component, represented by the stationary state of the traces. A magnification of the transient sodium current is also visible in the Figure insert. C. Representative current evoked by kainate. The kainate-induced currents were due to the activation of the AMPA receptors, since the response was reversibly blocked by the addition of the selective antagonist GYKI at 100 μM concentration. D. Example of current evoked by 200 μM kainate at a resting membrane potential of −60 mV, in a control cell with the empty vector and in an antisense cell at day 2 in vitro. The bar indicates the time of drug application. E. The histogram represents the mean of whole cell current amplitudes evoked by kainate administration under voltage clamp condition in function of the time in culture for the empty vector and antisense cells. The application of 200 μM kainate at the holding potential of −60 mV produced an inward current significantly greater (*p<0.05) in antisense neurons than in the empty vector neurons. This difference in current amplitude between empty vector and antisense cells, disappeared at 7 days in culture. F. Western blot of GluR2/3 subunits of AMPA receptor on differentiating NPC cultures at day 3. Densitometric analysis of the bands indicates a 2-fold increment in GluR2/3 subunits in Tβ4-antisense extracts, compared to control, using β-actin for normalisation. Values are represented as percentage of control. *p<0.01.

FIG. 9: Functional improvement of spinal cord injured mice grafted with Tβ4 antisense-NPCs.

(A) Upper panel: Analysis of locomotor function as assessed by the Catwalk system. Foot prints (left and right, fore and hind paws) digitized by the Catwalk software measuring the stride length, i.e. the distance between two consecutive prints on each side. Lower panel: Graphs showing the time course of right and left stride length in spinal cord injured mice that were grafted either with Tβ4 antisense-NPCs (n=4), or with Empty Vector-NPCs (n=5), or that were vehicle injected (n=5). Mice grafted with Tβ4 antisense-NPCs recovered their stride length better and faster (within 3 weeks after injury) than did mice grafted with Empty Vector-NPCs or vehicle-injected mice. Pre-injury values were collected one week before trauma during training sessions. (B) Graphs summarizing the results of locomotor assessment. Two out of seven parameters measured by the Catwalk system, intensity (R&L) and stride length (R&L), recovered significantly better in mice grafted with Tβ4 antisense-NPCs than in mice grafted with Empty Vector-NPCs both at 4 and at 8 weeks after spinal cord injury (*, p<0.05; **, p<0.01). Dotted lines represent the range (mean±s.d.) obtained before surgery.

FIG. 10: Survival of T34 antisense-NPCs in the spinal cord of injured mice.

(A) Sagittal sections of spinal cords by 8 weeks after graft/injury stained with hematoxylin/eosin. Asterisks indicate the lesion scar. Lesioned spinal cords are bent because of the lack of tissue at the site of injury. Bar, 300 μm. (B) and (C). Low power magnification of triple-labeled confocal images of sagittal sections from lesioned spinal cords transplanted with Empty Vector- or Tβ4 antisense-NPCs, to illustrate the survival and distribution of transplanted NPCs (green) in the lesion, the GFAP immunoreactivity surrounding the lesion (blue), and nuclei (red). Tβ4 antisense-NPCs survive better in the lesioned area and form coherent clusters. Bar, 100 micrometres. (C) Each channel has been separated from the merged images better to distinguish the different labeling of the injured spinal cord sections; Bar, 50 micrometres. (D) A close-up view of the transplanted NPCs which are stained by the anti-GFP as evidenced by the yellow labeling of the merged image. Bar, 20 micrometres. (E) Staining with an anti-GFAP along with an anti-Tβ4 antibody, confirms, that in the injured spinal cords Tβ4 antisense-NPCs maintain the reduction in the level of the peptide compared with the Empty Vector-NPCs. The expression of a higher level of Tβ4 by the grafted Empty Vector-NPCs is, evidenced by the yellow/orange staining in the merged image. Bar, 100 micrometres.

FIG. 11: Grafted Tβ4 antisense-NPCs retain a potential to differentiate into neurons and over-express L1.

(A) High magnification images of transplanted Empty Vector- or Tβ4 antisense-NPCs (green) and nuclei (red). Tβ4 antisense-NPCs acquire a neuronal-like phenotype whereas Empty Vector-NPCs retain a rounded shape, 8 weeks after injury. Bar, 20 micrometres. (B) Close-up view of three examples of Tβ4 antisense-NPCs-derived neurons (green) which are stained with the neuronal marker β tubulin III, or MAP2, or Smi32 (red). By contrast, Empty Vector-NPCs remain rounded and do not express (3 tubulin III. Bar, 20 micrometres. (C). Confocal images of grafted/injured spinal cord sections labeled for β tubulin III (blue) and L1 (red). Each channel has been separated from the merged images better to distinguish the different labelling of the injured spinal cords. Tβ4 antisense-NPCs (green) colocalize with L1 staining, which is strongly over-expressed as compared with Empty Vector-NPCs. Moreover, a high density of β tubulin III fibres, in close association with the grafted Tβ4 antisense-NPCs, is evident (arrows). Bar, 20 micrometres.

FIG. 12: Grafted Tβ4 antisense-NPCs promote regeneration of β tubulin III-positive axons and sprouting of 5HT-positive fibres.

(A). Low power magnification confocal images of spinal cord sections showing the grafted Empty Vector- or Tβ4 antisense-NPCs (green) along with β tubulin III (red), and GFAP (blue) staining. Tβ4 antisense-NPCs are surrounded and contacted by a high density of β tubulin III-positive fibres (arrows) in the lesioned area, 8 weeks after injury. (B). Co-expression of the marker of growing axons GAP43 (blue) and β tubulin III (red) in spinal cord sections of mice grafted with Empty Vector- or Tβ4 antisense-NPCs (green). Note a robust GAP43 immunostaining in correspondence with β tubulin III-positive fibres (arrows) at the level of the lesioned site containing Tβ4 antisense-NPCs compared with the lesion site containing Empty Vector-NPCs, indicating that regeneration of host axons occurs 8 weeks after injury.

(C). Analysis of 5-HT immunoreactivity (red), along with GFAP (blue) of sagittal sections of mice grafted with Empty Vector- or Tβ4 antisense-NPCs (green) 8 weeks after injury. The figure reveals close association of grafted Tβ4 antisense-NPCs and 5-HT-positive fibres within the GFAP-negative lesioned site. Bars, 20 micrometres.

### REFERENCES

Andreassen P R, Lacroix F B, Lohez O D, Margolis R L (2001) Neither p21WAF1 nor 14-3-3sigma prevents G2 progression to mitotic catastrophe in human colon carcinoma cells after DNA damage, but p21WAF1 induces stable G1 arrest in resulting tetraploid cells. Cancer Res 61:7660-7668.
Bignami A, Eng L F, Dahl D, Uyeda C T (1972) Localization of the glial fibrillary acidic protein in astrocytes by immunofluorescence. Brain Res 43:429-435.
Bixby J L, Zhang R (1990) Purified N-cadherin is a potent substrate for the rapid induction of neurite outgrowth. J Cell Biol 110:1253-1260.
Blenis J (1993) Signal transduction via the MAP kinases: proceed at your own RSK. Proc Natl Acad Sci USA 90:5889-5892.
Bock-Marquette I, Saxena A, White M D, Dimaio J M, Srivastava D (2004) Thymosin beta4 activates integrin-linked kinase and promotes cardiac cell migration, survival and cardiac repair. Nature 432:466-472.
Bradke F, Dotti C G (1999) The role of local actin instability in axon formation. Science 283:1931-1934.
Bradke F, Dotti C G (2000) Establishment of neuronal polarity: lessons from cultured hippocampal neurons. Curr Opin Neurobiol 10:574-581.
Cano E, Mahadevan L C (1995) Parallel signal processing among mammalian MAPKs. Trends Biochem Sci 20:117-122.
Carpintero P, Anadon R, Diaz-Regueira S, Gomez-Marquez J (1999) Expression of thymosin beta4 messenger RNA in normal and kainate-treated rat forebrain. Neuroscience 90:1433-1444.
Cha H J, Jeong M J, Kleinman H K (2003) Role of thymosin beta4 in tumor metastasis and angiogenesis. J Natl Cancer Inst 95:1674-1680.
Chen H, Bernstein B W, Bamburg J R (2000) Regulating actin-filament dynamics in vivo. Trends Biochem Sci 25:19-23.
Chen Q, Chen T J, Letourneau P C, Costa Lda F, Schubert D (2005) Modifier of cell adhesion regulates N-cadherin-mediated cell-cell adhesion and neurite outgrowth. J Neurosci 25:281-290.
Choe J, Sun W, Yoon S Y, Rhyu I J, Kim E H, Kim H (2005) Effect of thymosin beta15 on the branching of developing neurons. Biochem Biophys Res Commun 331:43-49.
Choi S Y, Kim D K, Eun B, Kim K, Sun W, Kim H (2006) Anti-apoptotic function of thymosin-beta in developing chick spinal motoneurons. Biochem Biophys Res Commun 346:872-878.
da Silva J S, Dotti C G (2002) Breaking the neuronal sphere: regulation of the actin cytoskeleton in neuritogenesis. Nat Rev Neurosci 3:694-704.
Dathe V, Brand-Saberi B (2004) Expression of thymosin beta4 during chick development. Anat Embryol (Berl) 208:27-32.
Devineni N, Minamide L S, Niu M, Safer D, Verma R, Bamburg J R, Nachmias V T (1999) A quantitative analysis of G-actin binding proteins and the G-actin pool in developing chick brain. Brain Res 823:129-140.
Doherty P, Williams G, Williams E J (2000) CAMs and axonal growth: a critical evaluation of the role of calcium and the MAPK cascade. Mol Cell Neurosci 16:283-295.
Dunnett S B (1995) Functional repair of striatal systems by neural transplants: evidence for circuit reconstruction. Behav Brain Res 66:133-142.
Erickson-Viitanen S, Ruggieri S, Natalini P, Horecker B L (1983) Distribution of thymosin beta 4 in vertebrate classes. Arch Biochem Biophys 221:570-576.
Fernandez-Valle C, Gorman D, Gomez A M, Bunge M B (1997) Actin plays a role in both changes in cell shape and gene-expression associated with Schwann cell myelination. J Neurosci 17:241-250.
Fisher L J, Gage F H (1995) Novel therapeutic directions for Parkinson's disease. Mol Med Today 1:181-187.
Gage F H, Ray J, Fisher L J (1995) Isolation, characterization, and use of stem cells from the CNS. Annu Rev Neurosci 18:159-192.
Gallo G, Letourneau P C (2004) Regulation of growth cone actin filaments by guidance cues. J Neurobiol 58:92-102.
Gao X, Bian W, Yang J, Tang K, Kitani H, Atsumi T, Jing N (2001) A role of N-cadherin in neuronal differentiation of embryonic carcinoma P19 cells. Biochem Biophys Res Commun 284:1098-1103.
Glotzer M (2005) The molecular requirements for cytokinesis. Science 307:1735-1739.
Goldstein A L (2003) Thymosin beta4: a new molecular target for antitumor strategies. J Natl Cancer Inst 95:1646-1647.
Golla R, Philp N, Safer D, Chintapalli J, Hoffman R, Collins L, Nachmias V T (1997) Co-ordinate regulation of the cytoskeleton in 3T3 cells overexpressing thymosin-beta4. Cell Motil Cytoskeleton 38:187-200.
Gomez-Marquez J, Anadon R (2002) The beta-thymosins, small actin-binding peptides widely expressed in the developing and adult cerebellum. Cerebellum 1:95-102.
Grant D S, Rose W, Yaen C, Goldstein A, Martinez J, Kleinman H (1999) Thymosin beta4 enhances endothelial cell differentiation and angiogenesis. Angiogenesis 3:125-135.
Hall A K (1991) Differential expression of thymosin genes in human tumors and in the developing human kidney. Int J Cancer 48:672-677.
Hamill O P, Huguenard J R, Prince D A (1991) Patch-clamp studies of voltage-gated currents in identified neurons of the rat cerebral cortex. Cereb Cortex 1:48-61.
Hattan D, Nesti E, Cachero T G, Morielli A D (2002) Tyrosine phosphorylation of Kv1.2 modulates its interaction with the actin-binding protein cortactin. J Biol Chem 277:38596-38606.
Hockfield S, McKay R D (1985) Identification of major cell classes in the developing mammalian nervous system. J Neurosci 5:3310-3328.
Huang W Q, Wang B H, Wang Q R (2006) Thymosin beta4 and AcSDKP inhibit the proliferation of HL-60 cells and induce their differentiation and apoptosis. Cell Biol Int 30:514-520.
Huff T, Muller C S, Otto A M, Netzker R, Hannappel E (2001) beta-Thymosins, small acidic peptides with multiple functions. Int J Biochem Cell Biol 33:205-220.
Huff T, Rosorius O, Otto A M, Muller C S, Ballweber E, Hannappel E, Mannherz H G (2004) Nuclear localisation of the G-actin sequestering peptide thymosin beta4. J Cell Sci 117:5333-5341.
Iwai Y, Usui T, Hirano S, Steward R, Takeichi M, Uemura T (1997) Axon patterning requires DN-cadherin, a novel neuronal adhesion receptor, in the Drosophila embryonic CNS. Neuron 19:77-89.
Kiryushko D, Berezin V, Bock E (2004) Regulators of neurite outgrowth: role of cell adhesion molecules. Ann N Y Acad Sci 1014:140-154.
Kobayashi T, Okada F, Fujii N, Tomita N, Ito S, Tazawa H, Aoyama T, Choi S K, Shibata T, Fujita H, Hosokawa M (2002) Thymosin-beta4 regulates motility and metastasis of malignant mouse fibrosarcoma cells. Am J Pathol 160:869-882.
Lambrechts A, Jonckheere V, Peleman C, Polet D, De Vos W, Vandekerckhove J, Ampe C (2006) Profilin-I-ligand interactions influence various aspects of neuronal differentiation. Cell Sci 119:1570-1578.
Letourneau P C, Shattuck T A (1989) Distribution and possible interactions of actin-associated proteins and cell adhesion molecules of nerve growth cones. Development 105:505-519.
Lin S C, Morrison-Bogorad M (1990) Developmental expression of mRNAs encoding thymosins beta 4 and beta 10 in rat brain and other tissues. J Mol Neurosci 2:35-44.
Luo L (2000) Rho GTPases in neuronal morphogenesis. Nat Rev Neurosci 1:173-180.
Malinda K M, Goldstein A L, Kleinman H K (1997) Thymosin beta 4 stimulates directional migration of human umbilical vein endothelial cells. Faseb J 11:474-481.
Malinda K M, Sidhu G S, Mani H, Banaudha K, Maheshwari R K, Goldstein A L, Kleinman H K (1999) Thymosin beta4 accelerates wound healing. J Invest Dermatol 113:364-368.
McGough A (1998) F-actin-binding proteins. Curr Opin Struct Biol 8:166-176.
Misonou H, Trimmer J S (2004) Determinants of voltage-gated potassium channel surface expression and localization in Mammalian neurons. Crit. Rev Biochem Mol Biol 39:125-145.
Moccia R, Chen D, Lyles V, Kapuya E, E Y, Kalachikov S, Spahn C M, Frank J, Kandel E R, Barad M, Martin KC (2003) An unbiased cDNA library prepared from isolated Aplysia sensory neuron processes is enriched for cytoskeletal and translational mRNAs. J Neurosci 23:9409-9417.
Moon H S, Even-Ram S, Kleinman H K, Cha H J (2006) Zyxin is upregulated in the nucleus by thymosin beta4 in SiHa cells. Exp Cell Res 312:3425-3431.
Muller C S, Huff T, Hannappel E (2003) Reduction of thymosin beta4 and actin in HL60 cells during apoptosis is preceded by a decrease of their mRNAs. Mol Cell Biochem 250:179-188.
Nuriya M, Huganir R L (2006) Regulation of AMPA receptor trafficking by N-cadherin. J Neurochem 97:652-661.
Olson L (1997) Regeneration in the adult central nervous system: experimental repair strategies. Nat Med 3:1329-1335.
Otero A, Bustelo X R, Pichel J G, Freire M, Gomez-Marquez J (1993) Transcript levels of thymosin beta 4, an actin-sequestering peptide, in cell proliferation. Biochim Biophys Acta 1176:59-63.
Otero J J, Fu W, Kan L, Cuadra A E, Kessler J A (2004) Beta-catenin signaling is required for neural differentiation of embryonic stem cells. Development 131:3545-3557.
Pang L, Sawada T, Decker S J, Saltiel A R (1995) Inhibition of MAP kinase kinase blocks the differentiation of PC-12 cells induced by nerve growth factor. J Biol Chem 270:13585-13588.
Perron J C, Bixby J L (1999) Distinct neurite outgrowth signaling pathways converge on ERK activation. Mol Cell Neurosci 13:362-378.
Petrecca K, Miller D M, Shrier A (2000) Localization and enhanced current density of the Kv4.2 potassium channel by interaction with the actin-binding protein filamin. J Neurosci 20:8736-8744.
Philp D, Goldstein A L, Kleinman H K (2004) Thymosin beta4 promotes angiogenesis, wound healing, and hair follicle development. Mech Ageing Dev 125:113-115.
Pollard T D (2003) The cytoskeleton, cellular motility and the reductionist agenda. Nature 422:741-745.
Pollard T D, Blanchoin L, Mullins R D (2001) Actin dynamics. J Cell Sci 114:3-4.
Redell J B, Xue-Bian J J, Bubb M R, Crow T (2007) One-trial in vitro conditioning regulates an association between the beta-thymosin repeat protein Csp24 and actin. Neuroscience 148:413-420.
Reynolds B A, Weiss S (1992) Generation of neurons and astrocytes from isolated cells of the adult mammalian central nervous system. Science 255:1707-1710.
Ricci-Vitiani L, Pedini F, Mollinari C, Condorelli G, Bonci D, Bez A, Colombo A, Parati E, Peschle C, De Maria R (2004) Absence of caspase 8 and high expression of PED protect primitive neural cells from cell death. J Exp Med 200:1257-1266.
Roth L W, Bormann P, Wiederkehr C, Reinhard E (1999a) Beta-thymosin, a modulator of the actin cytoskeleton is increased in regenerating retinal ganglion cells. Eur J Neurosci 11:3488-3498.
Roth L W, Bormann P, Bonnet A, Reinhard E (1999b) beta-thymosin is required for axonal tract formation in developing zebrafish brain. Development 126:1365-1374.
Safer D, Nachmias V T (1994) Beta thymosins as actin binding peptides. Bioessays 16:473-479.
Saffell J L, Williams E J, Mason I J, Walsh F S, Doherty P (1997) Expression of a dominant negative FGF receptor inhibits axonal growth and FGF receptor phosphorylation stimulated by CAMs. Neuron 18:231-242.
Sanger J M, Golla R, Safer D, Choi J K, Yu K R, Sanger J W, Nachmias V T (1995) Increasing intracellular concentrations of thymosin beta 4 in PtK2 cells: effects on stress fibers, cytokinesis, and cell spreading. Cell Motil Cytoskeleton 31:307-322.
Sosne G, Siddiqi A, Kurpakus-Wheater M (2004) Thymosin-beta4 inhibits corneal epithelial cell apoptosis after ethanol exposure in vitro. Invest Ophthalmol V is Sci 45:1095-1100.
Sosne G, Qiu P, Christopherson P L, Wheater M K (2007) Thymosin beta 4 suppression of corneal NFkappaB: a potential anti-inflammatory pathway. Exp Eye Res 84:663-669.
Sosne G, Szliter E A, Barrett R, Kernacki K A, Kleinman H, Hazlett L D (2002) Thymosin beta 4 promotes corneal wound healing and decreases inflammation in vivo following alkali injury. Exp Eye Res 74:293-299.
Stossel T P (1989) From signal to pseudopod. How cells control cytoplasmic actin assembly. J Biol Chem 264:18261-18264.
Sun H Q, Yin H L (2007) The beta-thymosin enigma. Ann N Y Acad Sci 1112:45-55.
Sweatt J D (2001) The neuronal MAP kinase cascade: a biochemical signal integration system subserving synaptic plasticity and memory. J Neurochem 76:1-10.
Takeichi M (1995) Morphogenetic roles of classic cadherins. Curr Opin Cell Biol 7:619-627.
Tanaka E, Sabry J (1995) Making the connection: cytoskeletal rearrangements during growth cone guidance. Cell 83:171-176.
Tanaka H, Shan W, Phillips G R, Arndt K, Bozdagi O, Shapiro L, Huntley G W, Benson D L, Colman DR (2000) Molecular modification of N-cadherin in response to synaptic activity. Neuron 25:93-107.
Theriot J A (1994) Regulation of the actin cytoskeleton in living cells. Semin Cell Biol 5:193-199.
Utsugisawa K, Nagane Y, Obara D, Tohgi H (2002) Overexpression of alpha7 nicotinic acetylcholine receptor prevents G1-arrest and DNA fragmentation in PC12 cells after hypoxia. J Neurochem 81:497-505.
Utton M A, Eickholt B, Howell F V, Wallis J, Doherty P (2001) Soluble N-cadherin stimulates fibroblast growth factor receptor dependent neurite outgrowth and N-cadherin and the fibroblast growth factor receptor co-cluster in cells. J Neurochem 76:1421-1430.
Uyeda T Q, Nagasaki A (2004) Variations on a theme: the many modes of cytokinesis. Curr Opin Cell Biol 16:55-60.
van Kesteren R E, Carter C, Dissel H M, van Minnen J, Gouwenberg Y, Syed N I, Spencer G E, Smit A B (2006) Local synthesis of actin-binding protein beta-thymosin regulates neurite outgrowth. J Neurosci 26:152-157.
Vescovi A L, Reynolds B A, Fraser D D, Weiss S (1993) bFGF regulates the proliferative fate of unipotent (neuronal) and bipotent (neuronal/astroglial) EGF-generated CNS progenitor cells. Neuron 11:951-966.
Wang W S, Chen P M, Hsiao H L, Ju S Y, Su Y (2003) Overexpression of the thymosin beta-4 gene is associated with malignant progression of SW480 colon cancer cells. Oncogene 22:3297-3306.
Wang W S, Chen P M, Hsiao H L, Wang H S, Liang W Y, Su Y (2004) Overexpression of the thymosin beta-4 gene is associated with increased invasion of SW480 colon carcinoma cells and the distant metastasis of human colorectal carcinoma. Oncogene 23:6666-6671.
Yamamoto M, Yamagishi T, Yaginuma H, Murakami K, Ueno N (1994) Localization of thymosin beta 4 to the neural tissues during the development of Xenopus laevis, as studied by in situ hybridization and immunohistochemistry. Brain Res Dev Brain Res 79:177-185.
Yamoah E N, Levic S, Redell J B, Crow T (2005) Inhibition of conditioned stimulus pathway phosphoprotein 24 expression blocks the reduction in A-type transient K+ current produced by one-trial in vitro conditioning of Hermissenda. J Neurosci 25:4793-4800.
Yap A S, Brieher W M, Gumbiner B M (1997) Molecular and functional analysis of cadherin-based adherens junctions. Annu Rev Cell Dev Biol 13:119-146.
Yu X, Malenka R C (2003) Beta-catenin is critical for dendritic morphogenesis. Nat Neurosci 6:1169-1177.
Zalvide J B, Alvarez C V, Vidal A, Dieguez C, Vega F V, Dominguez F (1995) Regulation of thymosin beta 4 mRNA levels during cell proliferation. Cell Prolif 28:85-91.

### FURTHER REFERENCES FOR IN VIVO WORK

Barbeau H, Rossignol S. Initiation and modulation of the locomotor pattern in the adult chronic spinal cat by noradrenergic, serotonergic and dopaminergic drugs. Brain Res 1991; 546: 250-60.
Camand E, Morel M P, Faissner A, Sotelo C, Dusart I. Long-term changes in the molecular composition of the glial scar and progressive increase of serotoninergic fibre sprouting after hemisection of the mouse spinal cord. Eur J Neurosci 2004; 20:1161-76.
Castellani V, De Angelis E, Kenwrick S, Rougon G. Cis and trans interactions of L1 with neuropilin-1 control axonal responses to semaphorin 3A. Embo J 2002; 21: 6348-57.
Chen J, Bernreuther C, Dihne M, Schachner M. Cell adhesion molecule 11-transfected embryonic stem cells with enhanced survival support regrowth of corticospinal tract axons in mice after spinal cord injury. J Neurotrauma 2005a; 22: 896-906.
Chen J, Wu J, Apostolova I, Skup M, Irintchev A, Kugler S, et al. Adeno-associated virus-mediated L1 expression promotes functional recovery after spinal cord injury. Brain 2007; 130: 954-69.
Chen S, Mantei N, Dong L, Schachner M. Prevention of neuronal cell death by neural adhesion molecules L1 and CHL1. J Neurobiol 1999; 38: 428-39.
Choe J, Sun W, Yoon S Y, Rhyu L I, Kim E H, Kim H. Effect of thymosin beta15 on the branching of developing neurons. Biochem Biophys Res Commun 2005; 331: 43-9.
Dong L, Chen S, Richter M, Schachner M. Single-chain variable fragment antibodies against the neural adhesion molecule CHL1 (close homolog of L1) enhance neurite outgrowth. J Neurosci Res 2002; 69: 437-47.
Dong L, Chen S, Schachner M. Single chain Fv antibodies against neural cell adhesion molecule L1 trigger L1 functions in cultured neurons. Mol Cell Neurosci 2003; 22: 234-47.
Fransen E, D'Hooge R, Van Camp G, Verhoye M, Sijbers J, Reyniers E, et al. L1 knockout mice show dilated ventricles, vermis hypoplasia and impaired exploration patterns. Hum Mol Genet 1998; 7: 999-1009.
Frisen J, Johansson C B, Torok C, Risling M, Lendahl U. Rapid, widespread, and longlasting induction of nestin contributes to the generation of glial scar tissue after CNS injury. J Cell Biol 1995; 131: 453-64.
Hamers F P, Koopmans G C, Joosten E A. CatWalk-assisted gait analysis in the assessment of spinal cord injury. J Neurotrauma 2006; 23: 537-48.
Hamers F P, Lankhorst A J, van Laar T J, Veldhuis W B, Gispen W H. Automated quantitative gait analysis during overground locomotion in the rat: its application to spinal cord contusion and transection injuries. J Neurotrauma 2001; 18: 187-201
Lemmon V, Farr K L, Lagenaur C. L1-mediated axon outgrowth occurs via a homophilic binding mechanism. Neuron 1989; 2: 1597-603.
Lindner J, Rathjen F G, Schachner M. L1 mono- and polyclonal antibodies modify cell migration in early postnatal mouse cerebellum. Nature 1983, 305:427-430
Namiki J, Tator C H. Cell proliferation and nestin expression in the ependyma of the adult rat spinal cord after injury. J Neuropathol Exp Neurol 1999; 58: 489-98.
Pallini R, Fernandez E, Gangitano C, Del Fa A, Olivieri-Sangiacomo C, Sbriccoli A. Studies on embryonic transplants to the transected spinal cord of adult rats. J Neurosurg 1989; 70: 454-62.
Pallini R, Fernandez E, Sbriccoli A. Retrograde degeneration of corticospinal axons following transection of the spinal cord in rats. A quantitative study with anterogradely transported horseradish peroxidase. J Neurosurg 1988; 68: 124-8.
Pallini R, Vitiani L R, Bez A, Casalbore P, Facchiano F, Di Giorgi Gerevini V, et al. Homologous transplantation of neural stem cells to the injured spinal cord of mice. Neurosurgery 2005; 57: 1014-25; discussion 1014-25.
Pearse D D, Pereira F C, Marcillo A E, Bates M L, Berrocal Y A, Filbin M T, et al. cAMP and Schwann cells promote axonal growth and functional recovery after spinal cord injury. Nat Med 2004; 10: 610-6.
Rathjen F G, Rutishauser U. Comparison of two cell surface molecules involved in neural cell adhesion. Embo J 1984; 3: 461-5.
Ribotta M G, Provencher J, Feraboli-Lohnherr D, Rossignol S, Privat A, Orsal D. Activation of locomotion in adult chronic spinal rats is achieved by transplantation of embryonic raphe cells reinnervating a precise lumbar level. J Neurosci 2000; 20: 5144-52.
Roonprapunt C, Huang W, Grill R, Friedlander D, Grumet M, Chen S, et al. Soluble cell adhesion molecule L1-Fc promotes locomotor recovery in rats after spinal cord injury. J Neurotrauma 2003; 20: 871-82.
Schreyer D J, Skene J H. Injury-associated induction of GAP-43 expression displays axon branch specificity in rat dorsal root ganglion neurons. J Neurobiol 1993; 24: 959-70.
Shibuya S, Miyamoto O, Auer R N, Itano T, Mori S, Norimatsu H. Embryonic intermediate filament, nestin, expression following traumatic spinal cord injury in adult rats. Neuroscience 2002; 114: 905-16.
Shihabuddin L S, Horner P J, Ray J, Gage F H. Adult spinal cord stem cells generate neurons after transplantation in the adult dentate gyrus. J Neurosci 2000; 20: 8727-35.
Sieber-Blum M, Schnell L, Grim M, Hu Y F, Schneider R, Schwab M E. Characterization of epidermal neural crest stem cell (EPI-NCSC) grafts in the lesioned spinal cord. Mol Cell Neurosci 2006; 32: 67-81.
Silver J, Miller J H. Regeneration beyond the glial scar. Nat Rev Neurosci 2004; 5: 146-56.
Xu G, Nie D Y, Wang W Z, Zhang P H, Shen J, Ang B T, et al. Optic nerve regeneration in polyglycolic acid-chitosan conduits coated with recombinant L1-Fc. Neuroreport 2004; 15: 2167-72.
Yan J, Welsh A M, Bora S H, Snyder E Y, Koliatsos V E. Differentiation and tropic/trophic effects of exogenous neural precursors in the adult spinal cord. J Comp Neurol 2004; 480: 101-14.
Yang H, Cheng X, Yao Q, Li J, Ju G. The promotive effects of thymosin beta4 on neuronal survival and neurite outgrowth by upregulating L1 expression. Neurochem Res 2008; 33: 2269-80.
Zhang Y, Bo X, Schoepfer R, Holtmaat A J, Verhaagen J, Emson P C, et al. Growth-associated protein GAP-43 and L1 act synergistically to promote regenerative growth of Purkinje cell axons in vivo. Proc Natl Acad Sci USA 2005; 102: 14883-8.

1. A neural progenitor cell for implanting in a patient, wherein the cell has been treated to reduce thymosin β4 (Tβ4) expression.

2. An NPC according to claim 1, which is from the same species as the patient.

3. An NPC according to claim 1, wherein the patient is human.

4. An NPC according to claim 1, wherein the NPC is obtained from the patient.

5. An NPC according to claim 1, wherein the NPC is obtained from the brain.

6. An NPC according to claim 5, wherein the NPC is obtained from the hippocampus, subventricular zone or olfactory bulb.

7. An NPC according to claim 1, wherein the NPC is cultured in a medium containing epidermal growth factor (EGF) and basic fibroblast growth factor (bFGF).

9. An NPC according to claim 1, wherein the treatment to reduce expression of Tβ4 comprises transfection of the NPC with antisense DNA.

10. An NPC according to claim claim 1, wherein the treatment to reduce expression of Tβ4 comprises transfection of the NPC with miRNA.

11. An NPC according to claim 1, wherein the treatment to reduce expression of Tβ4 comprises transfection of the NPC with antisense siRNA.

12. An NPC according to claim 9, wherein the DNA or RNA is comprised in a suitable expression plasmid or lentivirus.

13. An NPC according to claim 1, wherein the treatment of the NPC comprises transfection with an expressible and detectable marker linked with the means to reduce expression of Tβ4.

14. An NPC according to claim 1, wherein Tβ4 expression is reduced to less than 10% of normal.

15. An NPC according to claim 1, wherein Tβ4 expression is reduced to substantially undetectable levels.

16. An NPC according to claim 1, wherein the patient is to be treated for brain damage or a neurodegenerative disorder.

17. An NPC according to claim 1, wherein the neurodegenerative disorder is Alzheimer's, Parkinson's, Huntington's, Amyotrophic Lateral Sceloris (ALS).

18. An NPC according to claim 1, wherein the brain damage is due to stroke or is a spinal cord injury.

19. An NPC according to claim 1, wherein the NPCs are incubated with the treatment in the presence of EGF and bFGF.

20. (canceled)

21. A method for the treatment of a patient requiring neuroregeneration comprising administering an NPC as defined in claim 1, to the area of the patient requiring neuroregeneration.
